(19) United States
(12) Patent Application Publication
(10) Pub. No.: US 2025/0049554 A1
(43) Pub. Date: Feb. 13, 2025

(54) DUAL HOOP LOW PROFILE EMBOLIC PROTECTION DEVICE AND SYSTEM

(71) Applicant: Transaortic Medical, Inc., Morgan Hill, CA (US)

(72) Inventors: Michael T. Carley, San Jose, CA (US); Nestor Benito Aganon, San Jose, CA (US); Isaac Han Joon Kim, San Jose, CA (US); Cindy My Thi Tran, San Jose, CA (US); Cener Espiritu Balosbalos, Castro Valley, CA (US); Adam Robert Tanner, Sun Lakes, AZ (US)

(21) Appl. No.: 18/799,478

(22) Filed: Aug. 9, 2024

Related U.S. Application Data
(60) Provisional application No. 63/518,434, filed on Aug. 9, 2023.

(51) Int. Cl. A61F 2/01

(52) U.S. Cl. CPC A61F 2/011; A61F 2002/018; A61F 2210/0014; A61F 2230/0065; A61F 2230/0069

(57) ABSTRACT

In some examples, an embolic protection system is provided for deploying a device in a diseased vessel of a vasculature, the embolic protection system comprising: an embolic protection device (EPD) including: an EPD shaft; a dual-hoop filter provided at a distal end of the EPD shaft, the dual-hoop filter including a distal hoop provided at a distal end of the dual-hoop filter, and a proximal hoop provided at a proximal end of the dual-hoop filter; and a filter actuator located at or towards a proximal end of the EPD, the filter actuator operable to selectively open or close the distal hoop and the proximal hoop of the dual-hoop filter.

### CLAIM OF PRIORITY

This patent application claims the benefit of priority, under 35 U.S.C. Section 119(e), to Carley et al, U.S. Provisional Patent Application Ser. No. 63/518,434, entitled “DUAL HOOP LOW PROFILE EMBOLIC PROTECTION DEVICE AND SYSTEM,” filed on Aug. 9, 2023 (Attorney Docket No. 5367.014PRV), which is hereby incorporated by reference herein in its entirety.

### TECHNICAL FIELD

The present application relates generally to devices for medical interventions conducted through vessels such as the major arteries or veins, and more particularly to devices with deployment configurations for conducting percutaneous procedures such as percutaneous valve replacement or other vascular or cardiac interventions.

### BACKGROUND

Treatment of native heart valves for conditions such as valvar regurgitation using percutaneous transcatheter procedures may involve advancing a catheter or device through the vasculature to the target native valve. The target native valve may be calcified or have other diseases such as unwanted plaque or thrombus attached to the native leaflets, annulus, or other anatomical regions adjacent the native valve. Rubbing, scraping or contact between the treatment catheter and the calcifications, plaque, or thrombus can result in undesirable separation of these materials from the tissue with subsequent embolization into other parts of the body. By dint of its large size, a large profile of a conventional treatment catheter or instrument can greatly exacerbate risk of an embolization. Embolization can result in serious complications including, but not limited to, ischemia, stroke, tissue damage, reduced lung function, among other complications.

Additionally, the vessels around the heart itself, such as the aorta, may also be diseased and have similar unwanted buildups of plaque, thrombus, calcium, and other deposits and advancing the catheter through the vessel can also result in unwanted separation of these materials from the vessel walls with embolization. The risk of separation is exacerbated when using conventional catheters, devices, or other high-profile conventional instruments.

It would therefore be desirable to either prevent separation of the plaques, thrombus, and calcium deposits from the native heart and adjacent vessels, and in situations where this does occur, capture or prevent the materials from embolizing in the patient.

### BRIEF DESCRIPTION OF THE DRAWINGS

To easily identify the discussion of any particular element or act, the most significant digit or digits in a reference number refer to the figure number in which that element is first introduced.

FIG. 1 shows various portions of a diseased aorta.

FIG. 2 shows a native aortic valve AV which illustrates some of the challenges of treating a cardiac valve.

FIG. 3 illustrates components of an example embolic protection system including an example embolic protection device.

FIG. 4 illustrates an example loading tool hub of an example embolic protection device.

FIG. 5 illustrates an example filter of an example embolic protection device.

FIG. 6 is a schematic view of an example embolic protection device with a filter in a deployed or open configuration.

FIG. 7 is a schematic view of an example embolic protection device with a filter in a withdrawn or closed configuration.

FIG. 8 is a schematic view of an example embolic protection device with a filter in a deployed or open configuration.

FIG. 9 is a schematic view of an example embolic protection device in a deployed configuration inside a human vasculature.

FIG. 10 is a schematic view of an example embolic protection device with a surgical device in a deployed configuration inside a human vasculature.

FIG. 11 is a schematic view of an example embolic protection device with a filter in an open configuration.

FIG. 12 includes schematic views illustrating aspects of a filter for an example embolic protection device.

FIG. 13 is a schematic view of a filter for an example embolic protection device.

FIG. 14 is a schematic view showing an example embolic protection device inside a loading tool with the filter supported by a dilator.

FIG. 15 is a schematic view of a filter for an example embolic protection device.

FIG. 16A and FIG. 16B show pictorial views of a filter dilator, according to an example embodiment.

FIG. 17A and FIG. 17B show sectional views of the filter dilator of FIG. 16A and FIG. 16B.

FIG. 18 shows a pictorial view of an atraumatic distal tip of a filter dilator, according to an example embodiment.

FIG. 19 and FIG. 20 show pictorial views of open and closed configurations of a filter, according to an example embodiment.

FIG. 21A to FIG. 21D show pictorial and sectional views of the loading tool 334, according to an example embodiment.

FIG. 22A and FIG. 22B show respective side and top pictorial views of an example expandable introducer.

FIG. 22C shows a pictorial view of a sheath dilator, according to an example embodiment.

FIG. 23A and FIG. 23B show respective pictorial views of loaded and deployed configurations of a sheath dilator and mesh sheath of an expandable introducer, according to example embodiments.

FIG. 24A and FIG. 24B show respective sectional and enlarged part-sectional views of an expandable introducer, according to an example embodiment.

FIGS. 25A-26K show various aspects and operations in the preparation and deployment of an embolic protection device and embolic protection system components for use in a medical procedure, according to example embodiments.

FIGS. 27A-27D show various aspects and operations in the deployment of an embolic protection device and embolic protection system in a medical procedure, according to an example embodiment.

FIGS. 28A-28B show schematic views of an example telescoping introducer sheath for an embolic protection system.

FIG. 29 shows a schematic view of an example accordion-type introducer sheath for an embolic protection system.

FIGS. 30A-30D illustrate components of an example embolic protection system including an example embolic protection device.

FIG. 31 show various aspects and operations in the preparation of an embolic protection device and embolic protection system components for use in a medical procedure, according to example embodiments.

FIGS. 32A-32L show various aspects and operations in the deployment of an embolic protection device and embolic protection system in a medical procedure, according to an example embodiment.

### DETAILED DESCRIPTION

The present disclosure describes use of the devices and methods disclosed herein during treatment in or adjacent the aorta. One of skill in the art will appreciate that this is not intended to be limiting and the devices and methods disclosed herein may be used in other anatomic regions of the body. Additionally, the present disclosure describes an embolic protection system which includes several components (e.g., a low-profile embolic protection device, an introducer, an introducer dilator, a loading tool, a dilator for the embolic protection device, and so forth). In some examples, the embolic protection system can be deployed to install a therapeutic device such as a replacement prosthetic valve in a human heart. Other uses and applications are possible in a variety of human subjects. These components may be used all together as a kit, or they may be provided individually and used individually, or they may be provided and used in any combination.

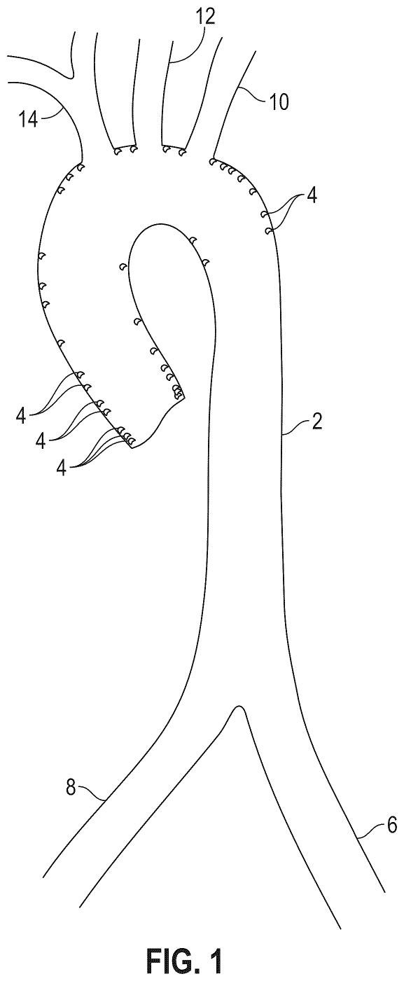

Referring to FIG. 1, an illustrative representation of a diseased aorta (2) is shown with deposits (4) distributed in several locations, including adjacent or within the left (6) and right (8) iliac arteries, and adjacent the junctions of the aortic arch with the left subclavian (10), left common carotid (12), and innominate artery (14). Navigating a diseased aorta (2) such as the one depicted in FIG. 1 is a challenge with conventional intravascular diagnostic and/or interventional hardware.

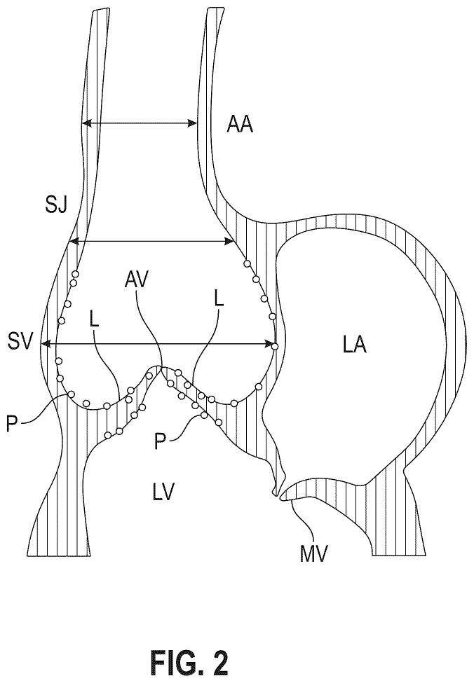

FIG. 2 shows a native aortic valve (AV) which illustrates some of the challenges of treating a cardiac valve. Here, the aortic valve (AV) includes native valve leaflets (L) which normally oppose one another to close the valve during contraction of the left ventricle (LV) to force blood away from the heart via the ascending aorta (AA). The aorta adjacent the aortic valve includes the sinus of Valsalva (SV) and the Sino-tubular junction (SJ). Blood is directed from the left atrium (LA) via the mitral valve (MV) into the left ventricle (LV). The native leaflets and walls of the aorta may be covered or lined with discrete or diffuse plaques (P) of material such as calcium, lipids, thrombus, or other unwanted materials. When a diagnostic or therapeutic device is advanced through the vasculature toward the aortic valve, the device may scrape or otherwise contact the plaques (P) and cause the plaques to separate from the tissue and then embolize downstream into other parts of the body and cause complications including ischemia, stroke, and so forth. It would therefore be desirable to provide methods and devices that prevent the unwanted separation of plaques from the native valve, aorta, or other adjacent tissue that could embolize, and in the event that embolization does occur, it would be desirable if such methods and devices capture the emboli and prevent them from flowing downstream. While this example emphasizes the aortic valve, one of skill in the art will appreciate that this is not limiting and the devices and methods disclosed herein may be used in other heart valves such as the mitral or tricuspid valve, or they may be used in other valves of the body including venous valves, or other organs and anatomical structures in the body.

In some examples, an embolic protection system is provided for deploying a device in a diseased vessel of a vasculature, the embolic protection system comprising: an embolic protection device (EPD) including: an EPD shaft; a dual-hoop filter provided at a distal end of the EPD shaft, the dual-hoop filter including a distal hoop provided at a distal end of the dual-hoop filter, and a proximal hoop provided at a proximal end of the dual-hoop filter; and a filter actuator located at or towards a proximal end of the EPD, the filter actuator operable to selectively open or close the distal hoop and the proximal hoop of the dual-hoop filter.

In some examples, the embolic protection system further comprises a dilator.

In some examples, the dilator includes an expandable tip.

In some examples, the embolic protection system further comprises a loading tool.

In some examples, the embolic protection system further comprises an introducer and/or an introducer sheath. In some examples, the introducer or introducer sheath is expandable. In some examples, the introducer or introducer sheath is non-expandable.

In some examples, there is provided a method of deploying an embolic protection device (EPD) into a vasculature of a patient, the method comprising: establishing access to the vasculature; introducing a guide wire into the vasculature to assist with guidance of the embolic protection device through the vasculature; advancing the embolic protection device into the vasculature and over the guide wire toward a target treatment region, wherein the embolic protection device comprises an EPD shaft, a dual-hoop filter provided at a distal end of the EPD shaft, the dual-hoop filter including a distal hoop provided at a distal end of the dual-hoop filter, and a proximal hoop provided at proximal end of the dual-hoop filter, and a filter actuator located at or towards a proximal end of the EPD, the filter actuator operable to selectively open or close the distal hoop and the proximal hoop of the dual-hoop filter; actuating the filter actuator thereby radially expanding at least one of the distal and proximal hoops of the dual-hoop filter to lie against a vessel wall at the target treatment region; and capturing emboli in the dual-hoop filter.

In some examples, the method further comprises inserting a therapeutic device through the filter and advancing the therapeutic device toward the target treatment region.

In some examples, the target treatment region comprises an aortic valve.

In some examples, the method further comprises, after a withdrawal of the therapeutic device through the filter, closing at least one of the distal hoop and the proximal hoop of the dual-hoop filter.

In some examples, an embolic protection device (EPD) comprises an EPD shaft; a dual-hoop filter provided at a distal end of the EPD shaft, the dual-hoop filter including a distal hoop provided at a distal end of the dual-hoop filter, and a proximal hoop provided at proximal end of the dual-hoop filter; and a filter actuator located at or towards a proximal end of the EPD, the filter actuator operable to selectively open or close the distal hoop and the proximal hoop of the dual-hoop filter.

In some examples, the dual-hoop filter includes a polymer mesh.

In some examples, the dual-hoop filter includes a particulate entrapment feature.

In some examples, the particulate entrapment feature defines a passage or gap in the dual-hoop filter allowing a therapeutic device to pass therethrough when the therapeutic device is passed through the distal hoop and the proximal hoop of the dual-hoop filter.

In some examples, at least one of the distal hoop and the proximal hoop is formed integrally with an actuation wire extending between the dual-hoop filter and the filter actuator.

In some examples, the filter actuator includes a distal slider to operate the distal hoop, and a proximal slider to operate the proximal hoop.

In some examples, the filter actuator includes a single slider to operate the distal hoop and the proximal hoop simultaneously.

In some examples, at least one of the distal hoop and the proximal hoop includes nitinol material and is pre-shaped to open in a round or oval configuration to conform to an aortic wall at a target treatment region.

In some examples, a central portion of the dual-hoop filter, when opened, has a lower profile than both end portions of the dual-hoop filter when the distal hoop and the proximal hoop are open.

In some examples, the EPD shaft includes a continuous braided polymer extrusion.

In some examples, the EPD shaft is a composite shaft and includes a distal shaft and a proximal shaft.

In some examples, at least one of the distal hoop and the proximal hoop is an offset hoop.

Referring to the accompanying drawings, various aspects of deployment steps and configurations utilizing examples of the present embolic protection devices and systems are now described.

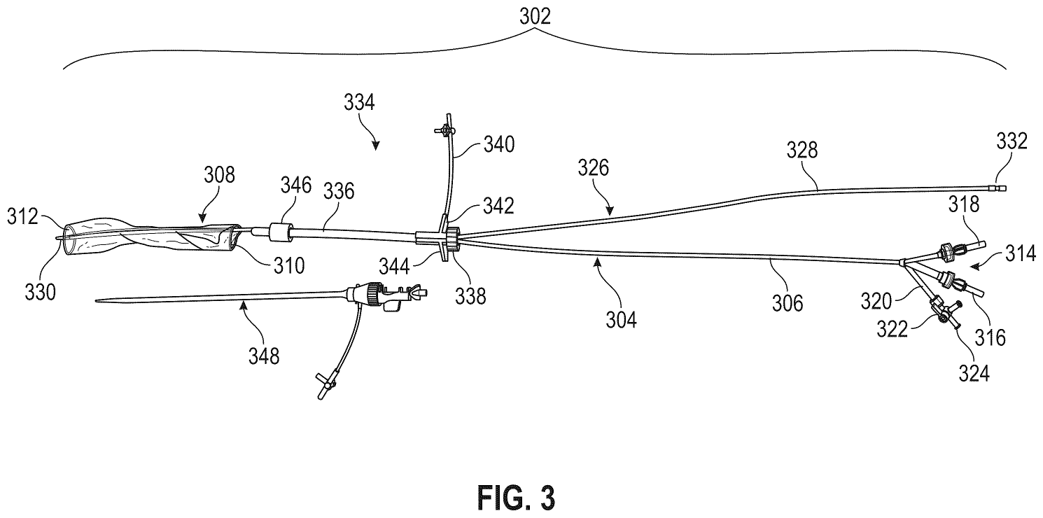

With reference to FIG. 3, examples of an embolic protection system 302 may include an embolic protection device 304 (also known as an EPD) that, viewed broadly, includes an EPD shaft 306, a filter 308 disposed at a distal end of the EPD shaft 306, and an EPD hub 314 disposed at a proximal end of the EPD shaft 306.

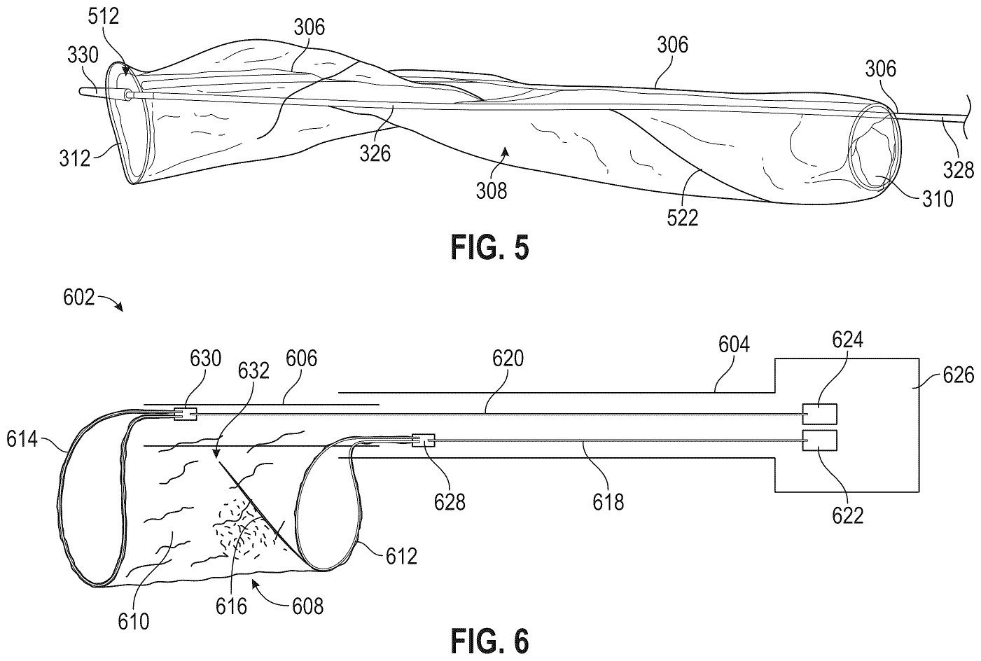

The filter 308 is supported on a distal end of the embolic protection device 304 and includes a proximal hoop 310 and a distal hoop 312 described more fully below. The filter 308 includes a flexible mesh material to capture and retain embolic particulates and other matter. With reference to FIG. 5, the filter 308 may include one or more internal portions or retainers, such as a particulate funnel 522.

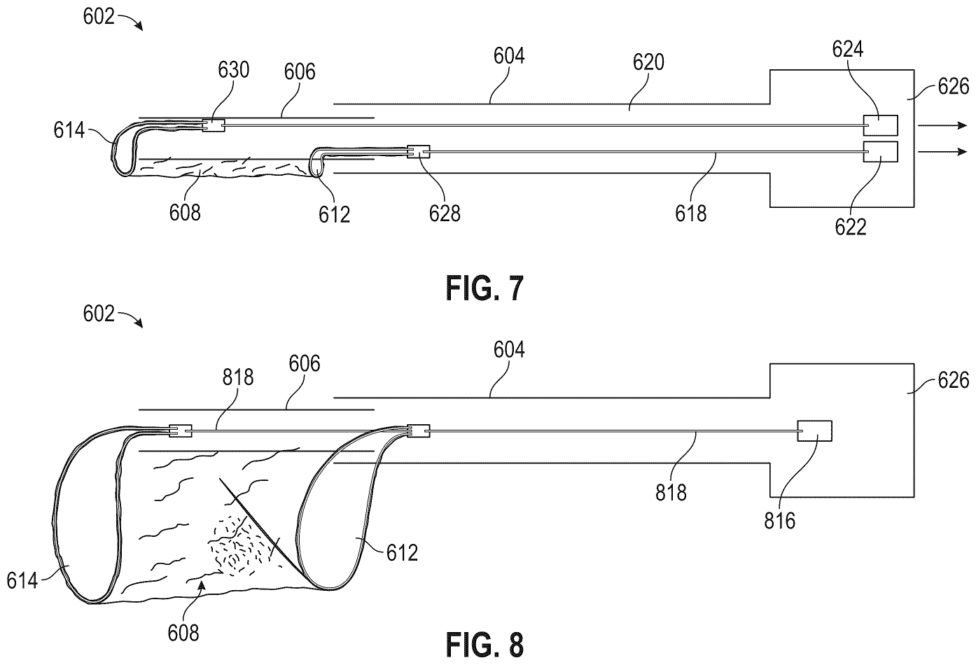

The EPD hub 314 includes a proximal hoop controller 316 and a distal hoop controller 318. The proximal hoop controller 316 (or actuator) may include a manually operable slider that is connected by a proximal actuation wire (not shown in FIG. 3, but see for example FIG. 6 or FIG. 7) extending through the EPD shaft 306 to the proximal hoop 310. The proximal hoop controller 316 can be manipulated by an operator to control the proximal hoop 310, for example to open or close the proximal hoop 310 and thereby open or close a proximal end of the filter 308.

The distal hoop controller 318 (or actuator) may include a manually operable slider that is connected by a distal actuation wire (not shown in FIG. 3, but see for example FIG. 6 or FIG. 7) extending through the EPD shaft 306 to the distal hoop 312. The distal hoop controller 318 can be manipulated by an operator to control the distal hoop 312, for example to open or close the distal hoop 312 and thereby open or close a distal end of the filter 308.

The EPD hub 314 further comprises an EPD flushing port 320. The EPD flushing port 320 includes a stopcock 322 (or valve) and one or more flushing connections 324.

In some examples, the embolic protection system 302 also comprises a filter dilator 326 to assist in deploying the embolic protection device 304 and the filter 308. The filter dilator 326 includes a filter dilator shaft 328, an atraumatic filter dilator tip 330 located at a distal end of the filter dilator 326, and a filter dilator flushing port 332 (or Luer) located at a proximal end of the filter dilator 326. The filter dilator 326 can pass through the proximal hoop 310 and into the filter 308 for example as shown in FIG. 3, and in enlarged view in FIG. 5. When needed, the distal hoop 312 can be closed by operating the distal hoop controller 318 to engage with the atraumatic filter dilator tip 330 as described more fully below. In some examples, the distal hoop 312 may engage with or be locked in a channel in the atraumatic filter dilator tip 330 as described further below. See for example the filter dilator channel 512 in FIG. 5. Some examples may not include a filter dilator channel 512, or a formation defining a filter dilator channel 512, for reasons of improved or lower profile of the embolic protection device 304.

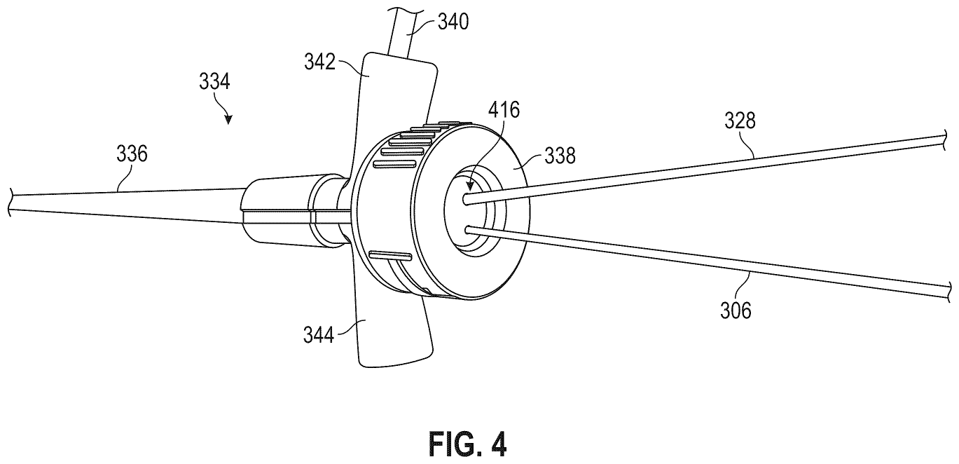

In some examples, the embolic protection system 302 further comprises a loading tool 334. The EPD shaft 306 of the embolic protection device 304 and the filter dilator shaft 328 of the filter dilator 326 can pass through or be withdrawn from sealed apertures in a loading tool hub 338 of the loading tool 334 (for example see the sealed apertures 416 in the loading tool hub 338 of FIG. 4) in readiness for deployment. In some examples, the loading tool 334 includes a loading sheath 336, a loading tool hub 338, a loading tool flushing line 340, and a pair of peel away arms or parts (for example peel away arm 342 and peel away arm 344) that can be manipulated to separate or split the loading sheath 336 to peel the loading tool 334 away from i.e., remove it from the embolic protection device 304 and/or the filter dilator 326, as described more fully below. In some examples, the loading tool may not be a peel away or include a peel away configuration and may remain in place during use.

In some examples, the embolic protection system 302 further comprises an expandable introducer 348 described more fully below. The embolic protection system may also comprise a fixed diameter introducer (not an expandable introducer).

Reference is now made to FIG. 6 of the accompanying drawings which shows a schematic view of an example “low-profile” embolic protection device 602, according to an example of the present disclosure. In this application, examples of “low-profile” embolic protection devices are also referred to simply as “embolic protection devices”. In some examples, such as in the illustrated example, the embolic protection device 602 is provided as a component of an embolic protection system, such as the embolic protection system 302 of FIG. 3.

As shown further in FIG. 6, the embolic protection device 602 generally comprises a proximal shaft 604 (or first flexible spine) connected to a distal shaft 606 (or second flexible spine). The embolic protection device 602 includes a filter 608 supported by the distal shaft 606.

The filter 608 includes a polymer mesh 610. The filter 608 and/or the polymer mesh 610 may include an entrapment feature 616. In some examples, one or more entrapment features is provided. An entrapment feature 616 can capture embolic material and other particulate material. The entrapment feature 616 may include a funnel or funnel-shaped feature as shown. The entrapment feature 616 is configured to leave a gap 632 or opening in the filter 608 that allows devices (such as a Transcatheter Aortic Valve Replacement device, or TAVR device) to pass through the filter 608, but still present a barrier or receptacle to embolic or other particulate material. The entrapment feature 616 may be formed integrally with the polymer mesh 610, such as being sewn into the polymer mesh 610, but other configurations of a filter 608 and polymer mesh 610 are possible, for example as described further below. In some examples, a proximal end of the entrapment feature 616 is permanently closed and is fixed to a proximal location of the filter 608 and/or the polymer mesh 610, as shown in FIG. 6. Other fixture locations are possible. The closed proximal end of the entrapment feature 616 allows capture but also maintains retention of embolic material while the filter 608 is being repositioned or withdrawn from a surgical site, for example.

The filter 608 includes a proximal hoop 612 located at a proximal end of the filter 608 and a distal hoop 614 located at a distal end of the filter 608. The proximal hoop 612 is connected to a proximal actuation wire 618 by a proximal actuation wire connector 628. The distal hoop 614 is connected to a distal actuation wire 620 by a distal actuation wire connector 630. A continuous actuation wire (or wires), or a single actuation wire operating both hoops, is possible for example as shown in FIG. 7 and FIG. 8. In the view of FIG. 6, the filter 608 is shown in an open or expanded configuration. Either end of the filter 608 can be opened or closed by manipulation of actuation sliders described more fully below. In some examples, the filter 608 is provided as a closable basket or net. The filter 608 includes braided filter material, or a mesh or net filter material such as polymer mesh 610 to capture (when open) and retain (when closed) embolic and other waste materials, such as plaque flakes and blood clots. The polymer mesh 610 may have a porosity that permits blood or other fluids to pass through the filter while retaining the embolic or other materials. Polyester, polyurethane, nylon, nitinol, or other materials may be used for the polymer mesh 610. The filter 608 and/or the polymer mesh 610 may be coated with heparin, hydrophilic polymer, silicone, or another material to prevent platelet adhesion and/or increase lubricity.

As shown in FIG. 6, the proximal actuation wire 618 is connected to a proximal slider 622 provided in a handle 626 (or filter actuator). The distal actuation wire 620 is connected to a distal slider 624 also provided in the handle 626. Manual operation or manipulation of the proximal slider 622 and the distal slider 624 can open and close the proximal hoop 612 and the distal hoop 614, respectively. A distal mouth or proximal mouth of the filter 608 can be opened and closed by an operator manipulating the proximal slider 622 and the distal slider 624 in the handle 626. When manipulated, movement of an actuation slider serves to apply tension to or relieve tension from the respective actuation wire to which it is connected. Corresponding movement of the actuation wire in turn opens and/or closes the distal or proximal mouth of the filter 608, as desired. Such operation of the distal and proximal hoops or manipulation of the sliders can form part of methods of treatment as described more fully below, particularly in relation to capture and retention of embolic and other particulate material generated during surgical procedures.

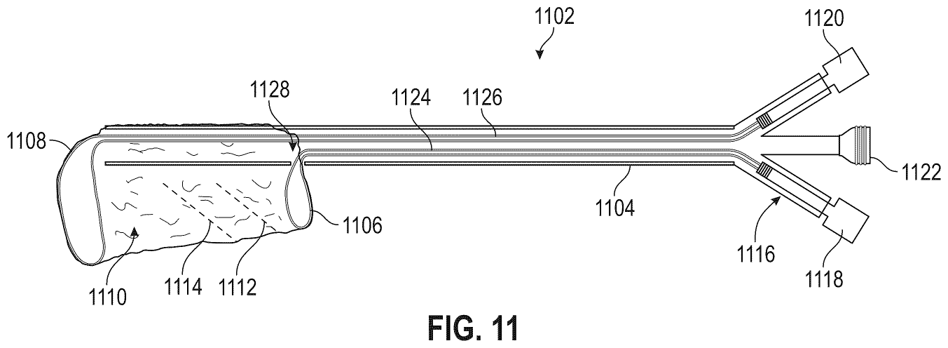

In some examples, the proximal shaft 604 and the distal shaft 606 are hollow and in conjunction support the filter 608 in use. In some examples, the proximal shaft 604 and the distal shaft 606 are provided in one piece or formed integrally, for example as shown in FIG. 11. In some examples, the proximal shaft 604 and/or the distal shaft 606 include a nitinol material and/or a stainless steel braided polymer material. Other materials are possible such as polyethylene, polypropylene, a fluorinated polymer, and a polyurethane, for example.

In some examples, the proximal shaft 604 and/or the distal shaft 606 are each very slender (needle-like) and highly flexible, having an outer diameter in the range 0.020-0.070 inches, and an inner diameter in the range 0.010-0.040 inches. The small outer dimensions of the proximal shaft 604 and the distal shaft 606 contribute to the “low-profile” characteristic of the embolic protection device 602. In some examples, a usable length of the proximal shaft 604 and the distal shaft 606 (and for other variations of a single or composite EPD shaft described herein) between the handle 626 and the proximal end of the filter 608 is in the range 40-120 cm but this length may be shorter or longer, as may be desired to suit different sizes of human anatomy or applications.

In some examples, the filter 608 includes a part frustoconical shape when open substantially as shown in FIG. 6. Other shapes and configurations of the filter 608 are possible. In some examples, as described further below, a shape or configuration of the filter 608 allows it to assume a very compact elongated tubular or snake-like form when collapsed, for example when being deployed, repositioned or withdrawn from a surgical site.

In some examples, the hoop wire of the proximal hoop 612 and/or the distal hoop 614 is formed integrally as an extension of an actuation wire. In some examples, the hoop wire is formed as a separate hoop, for example as shown in FIG. 6. The distal mouth and/or proximal mouth of the filter 608 may include, or be attached to, self-expanding members to bias the distal mouth and/or proximal mouth to an open or closed configuration. In some examples, a distal or proximal mouth of the filter may include, or be attached to, self-contracting members to bias the distal or proximal mouth to a closed configuration.

In FIG. 7, the distal hoop 614 is closed and the proximal hoop 612 is closed. The filter 608 is closed and lies alongside the distal shaft 606. The filter 608 assumes a narrow compact form as described above. The proximal slider 622 and the distal slider 624 have been moved back to close the respective hoops.

In FIG. 8, a single slider 816 can be manipulated to open and close both hoops at the same time. A single actuation wire 818 provided in one or more sections or extensions as shown connects the single slider 816 to the proximal hoop 612 and the distal hoop 614. The single slider 816 can be manipulated by an operator in advance and retract directions to control the proximal hoop 612 and the distal hoop 614 and open or close the hoops (or adopt positions in between full open and close) simultaneously.

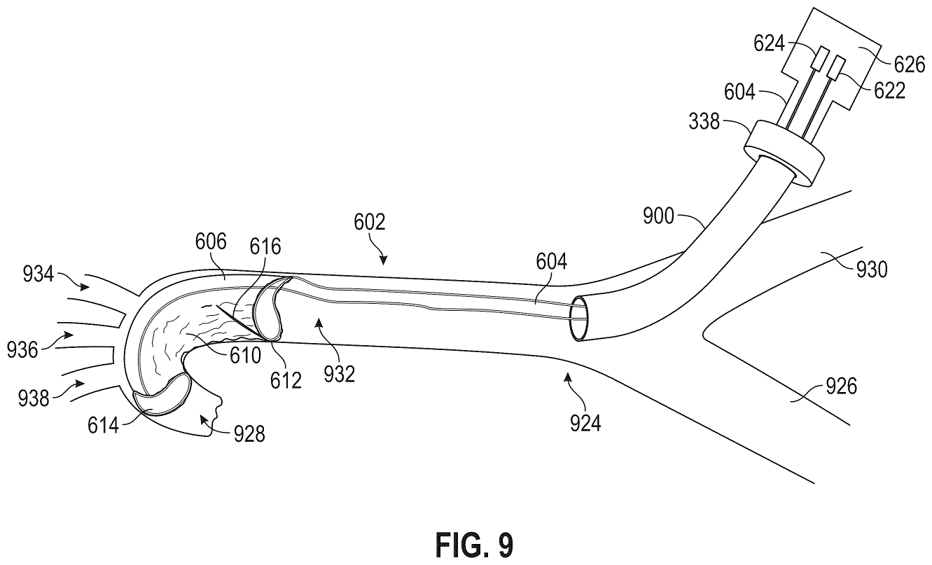

FIG. 9 is a schematic view of an example embolic protection device 602 introduced by an introducer sheath 900 into a human vasculature 924 and shown in a deployed configuration with the filter 608 open. Examples of the introducer sheath 900 and the loading tool 334 and methods of their use are described further below.

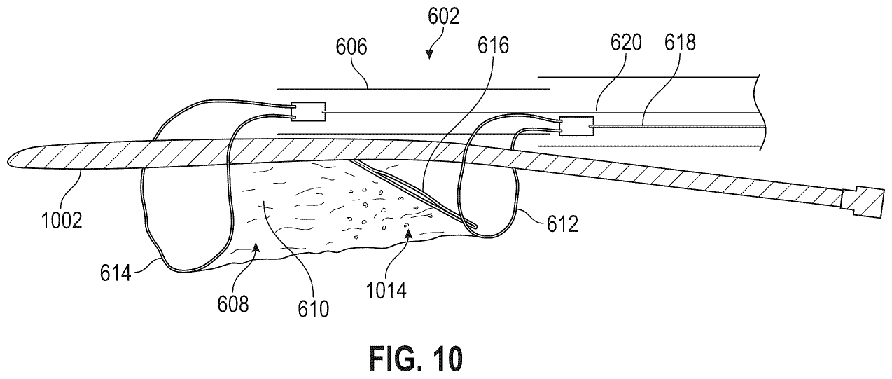

The human vasculature 924 generally includes the left iliac artery 926 and the right iliac artery 930. As illustrated, the embolic protection device 602 has been introduced into the right iliac artery 930, but introduction into of the embolic protection device 602 into the left iliac artery 926 is also possible. In some examples, a device such as a TAVR device (for example a TAVR device 1002 shown in FIG. 10) can be introduced into an artery opposite to that of the embolic protection device 602. The human vasculature 924 for example as illustrated also includes an ascending aorta 928 and a descending aorta 932. The left subclavian 934, the left common carotid 936 and the innominate artery 938 are shown adjacent the ascending aorta 928.

The embolic protection device 602 again generally comprises a proximal shaft 604 and a distal shaft 606 on which the filter 608 is supported. After introduction by the loading tool 334, the proximal slider 622 and the distal slider 624 can be manipulated to open the proximal hoop 612 and the distal hoop 614. The proximal hoop 612 is opened and brought into contact with the walls of the descending aorta 932. The distal hoop 614 is opened and brought into contact with the walls of the ascending aorta 928. During a surgical procedure, the filter 608 and polymer mesh 610 can capture (when open) and retain (when closed) embolic and other waste materials, such as plaque flakes and blood clots. Closure of the distal hoop 614 can protect, for example, embolic and other waste material from entering the ascending aorta 928, and/or the left subclavian 934, and/or the left common carotid 936, and/or the innominate artery 938 to assist in mitigating risk of stroke or embolism. Closure of the proximal hoop 612 can protect, for example, embolic and other waste material from entering the descending aorta 932 and assist in mitigating the risk of associated complications.

In FIG. 10, a TAVR device 1002 is shown passing through the filter 608 and entrapment feature 616 (for example, a funnel as shown) in a deployed configuration of the embolic protection device 602. Embolic debris 1014 has been captured by the entrapment feature 616. In this example, the proximal hoop 612 and the distal hoop 614 comprise tubular nitinol material and the hoops are pre-shaped to open (when the sliders are released) in a “round” configuration to conform to the aortic wall.

The polymer mesh 610 is attached around each hoop forming an internal passage. The filter 608 captures embolic debris 1014 but allows blood flow through the pores of the polymer mesh 610, while allowing access of the TAVR device 1002. After deployment, the proximal hoop 612 and the distal hoop 614 can be pulled or retracted into the distal shaft 606 using respective sliders and connected actuation wires. In some examples, the tapered funnel shape of the entrapment feature 616 can greatly assist in capturing embolic debris 1014 while allowing devices (such as the TAVR device 1002) to pass through the embolic protection device 602. Other arrangements and configurations of the embolic protection device 602, the filter 608, the polymer mesh 610, the proximal hoop 612, the distal hoop 614, and the entrapment feature 616 are possible to this end, for example as described below.

In FIG. 11, an example embolic protection device 1102 (EPD device) includes an EPD shaft 1104 extending between an EPD hub 1116 and a distal nitinol hoop 1108 of a filter 1110. In some examples, the EPD shaft 1104 is a unitary or one-piece EPD shaft. The filter 1110 includes one or more particle entrapment features, such as a first particulate capture feature 1112 and a second particulate capture feature 1114 which may (or may not) be funnel shaped or inclined at an angle to expected passage of embolic waste or debris. Other particulate capture features may be provided or are possible.

In this illustrated example, the distal nitinol hoop 1108 and a proximal nitinol hoop 1106 of the filter 1110 are each made from, and form part of, a long continuous nitinol wire. A long continuous wire may be provided for example as a first continuous nitinol wire 1124 and/or a second continuous nitinol wire 1126, as shown, to form integral distal and proximal nitinol hoops and respective nitinol actuation wires, accordingly. Here, both ends of the first continuous nitinol wire 1124 and the second continuous nitinol wire 1126 are passed through and extend inside the EPD shaft 1104 to respective actuation sliders, for example the proximal actuation slider 1118 and the distal actuation slider 1120 provided in the EPD hub 1116. A middle section of each first continuous nitinol wire 1124 and the second continuous nitinol wire 1126 forms a hoop to open and close proximal and distal ends of the filter 1110. For the proximal nitinol hoop 1106, the EPD shaft 1104 includes a proximal hoop opening 1128 to allow the first continuous nitinol wire 1124 to pass through to form the proximal nitinol hoop 1106.

In some examples, the EPD shaft 1104 is or includes a continuous braided polymer extrusion that runs from the distal nitinol hoop 1108 to the EPD hub 1116. The EPD shaft 1104 is extremely thin and flexible. In some examples, the EPD shaft 1104 is very slender (needle-like) and highly flexible, having an outer diameter in the range 0.020-0.070 inches, and an inner diameter in the range 0.010-0.040 inches. The small outer dimensions of the EPD shaft 1104 contribute to the “low-profile” characteristic of the embolic protection device 1102. In some examples, a usable length of the EPD shaft 1104 between the EPD hub 1116 and the proximal end of the filter 1110 is in the range 40-120 cm but this length may be shorter or longer, as may be desired to suit different sizes of human anatomy or applications.

In some examples, the thin, elongated, and extremely low profile configuration of the EPD shaft 1104 allows the embolic protection device 1102 to be introduced into a human vasculature 924 without a loading tool or expandable introducer mentioned above and described further below. The risk of generating embolic waste and debris during surgical procedures through the reduced use of tools may be mitigated accordingly in some examples. Prior to deployment, the EPD shaft 1104 can be flushed using a flushing port 1122, such as a Luer fitting.

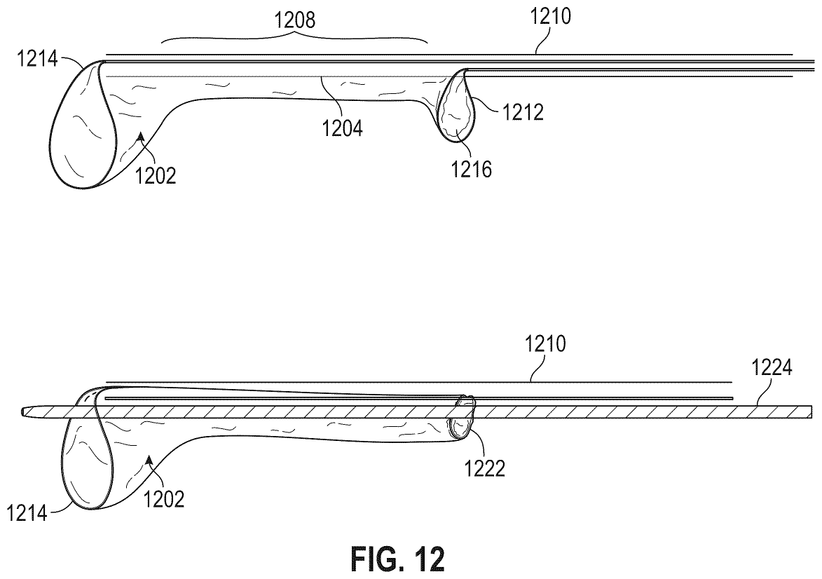

With reference to FIG. 12, further profile reduction features are now described. A filter 1202 for an embolic protection device may include reduced polymer mesh 1204 in a given section. For example, a middle or central, profile reduction section 1208 may be provided as shown. Other profile reduction sections of the filter 1202 are possible along the length of the EPD shaft 1210. When the filter 1202 is retracted into or alongside an EPD shaft 1210 (for example when the proximal hoop 1212 and the distal hoop 1214 are closed) the cross-sectional profile and bulk of the filter 1202 is significantly reduced accordingly. Despite the narrower central profile, embolic debris 1014 and waste can still be trapped safely in a region adjacent the proximal hoop 1212, as shown. To this end, additional filter mesh material 1222 is provided in some examples in the region of, or around, the proximal hoop 1212, as shown. This additional filter mesh material 1222 can help to capture embolic debris 1216 when the proximal hoop 1212 is closed over a TAVR device 1224 (or other device), for example. In some examples, the proximal hoop 1212 and/or the distal hoop 1214 can be spring loaded or otherwise biased (for example elastically) to close around the TAVR device 1224 (or other device) and then manually opened using an actuation slider for example. Other biasing configurations are possible.

The EPD shaft 1210 itself can be provided as an extremely thin-walled and fine nitinol tube having outer diameter (OD), inner diameter (ID) and wall thickness ranges as follows: OD 0.020-0.040 inches, ID 0.010-0.030 inches, wall thickness 0.002-0.010 inches. In some examples, the nitinol tube can be provided as a singular length for the EPD shaft 1210 or form part of a distal shaft in a composite EPD shaft (for example as a distal shaft 606 of FIG. 6). The thin nitinol material reduces profile and adds flexible support to the filter 1202. In some examples, the nitinol distal and/or proximal hoops are heat shaped into a round or oval shape.

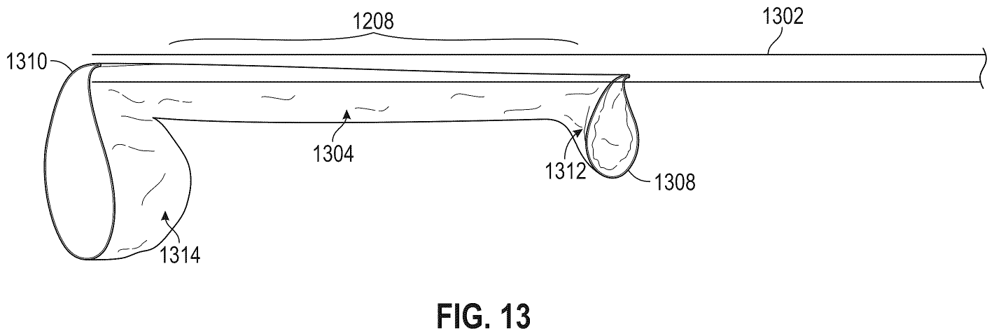

With reference to FIG. 13, an example filter 1304 supported on an EPD shaft 1302 is provided with one or more enlarged filter zones located adjacent a profile reduction section 1208. The enlarged zones can be provided in other filter sections. The enlarged zones may include, for example, a proximal filter pocket 1312 and/or a distal filter pocket 1314. The proximal filter pocket 1312 and/or distal filter pocket 1314 can also be provided adjacent to or include the proximal hoop 1308 and the distal hoop 1310 to define, in some examples, an openable and closable wide debris-capturing mouth, akin to a Manta ray by way of analogy. The illustrated example enjoys the benefit of having such a mouth at both ends of the filter 1304.

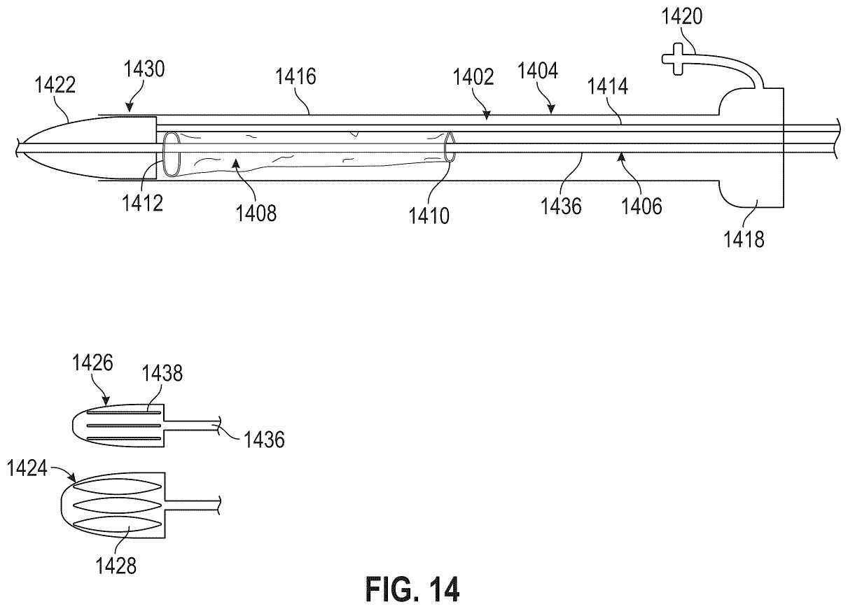

FIG. 14 is a schematic view showing an example embolic protection device 1402 located inside a loading tool 1404. For deployment, the filter 1408 of the embolic protection device 1402 can be engaged with or at least supported by a filter dilator 1406 extending though the filter 1408. The filter dilator 1406 includes a filter dilator shaft 1436. Examples of a filter dilator 1406 and methods of engagement and support during deployment are described further below. In the example illustrated here, the filter dilator 1406 has an expandable dilator tip 1422. In some examples, the expandable dilator tip is an atraumatic expandable dilator tip. The profile of the expandable dilator tip 1422 can be adjusted to suit a profile of the loading tool 1404, more specifically a leading end of an introducer sheath 1416 of the loading tool 1404, as shown for example at matching profile 1430. A matching profile can help to prevent undesirable snagging or uneven formations that might hinder introduction of the embolic protection device 1402 into the human vasculature.

The embolic protection device 1402 includes a proximal hoop 1410 and a distal hoop 1412 located at opposite ends of the filter 1408. The filter 1408 is supported on an EPD shaft 1414. The proximal hoop 1410 and the distal hoop 1412 can be opened and closed using actuation wires and sliders as described above, but omitted here in the interest of clarity. The loading tool 1404 includes the introducer sheath 1416, a loading tool hub 1418, and a flushing port 1420. The proximal hoop 1410 and the distal hoop 1412 can be compressed into the introducer sheath 1416 with the hoops open or closed in readiness for deployment of the embolic protection device 1402.

As mentioned above, for ease of introduction of the embolic protection device 1402 and the loading tool 1404 into the human vasculature (for example the human vasculature 924 of FIG. 9) the nose of the filter dilator 1406 is provided with an expandable dilator tip 1422. The expandable dilator tip 1422 can expand in a radial direction (for example as shown at 1424) under advancement of the expandable dilator tip 1422, but contract (for example as shown at 1426) when pulled in the opposite direction. An advancement may occur for example upon entry of the embolic protection device 1402 into the human vasculature i.e., when the filter dilator 1406 is under compression and being pushed by an operator. The expandable dilator tip 1422 can contract in a radial direction when the filter dilator 1406 is withdrawn (i.e., when under tension and being pulled by an operator).

In some examples, the expandable dilator tip 1422 may include slits that bulge open (see for example, open slits 1428 in the view of 1424) when the expandable dilator tip 1422 is compressed to expand the cross-sectional profile of the expandable dilator tip 1422 under axial compression, but narrow down (see for example closed slits 1438 in the view of 1426) when the filter dilator 1406 is pulled under tension to reduce the cross-sectional profile and allow radial contraction of the expandable dilator tip 1422. Other profile matching examples of an expandable dilator tip 1422 can include a deformable ball or flexible spherical structure that becomes oblate and fatter when compressed, but more oval in length and thinner when pulled. A thinner radially contracted profile of the expandable dilator tip 1422 can ease extraction of the filter dilator 1406 from the embolic protection device 1402 and the human vasculature.

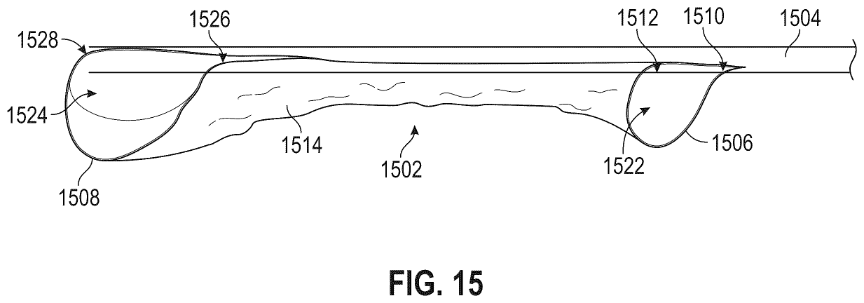

With reference to FIG. 15, an example filter configuration for further profile reduction in an embolic protection device is now described. A filter 1502 is supported on an EPD shaft 1504 and includes polymer mesh 1514. The filter 1502 includes a proximal hoop 1506 and a distal hoop 1508 that may be operated as described above. Other features of an embolic protection device are omitted here for clarity, but any one or more of such features could be incorporated into the illustrated example. Here, the proximal hoop 1506 and the distal hoop 1508 are “offset” hoops. An “offset” hoop can be compressed into a smaller loading sheath of a loading tool. Overlapping sections of the polymer mesh 1514 can be reduced or avoided entirely. For example, the proximal region 1522 and the distal region 1524 (bounded visually by their respective hoops in the view of FIG. 15) are each comprised of a single layer of the polymer mesh 1514 only. When compressed, these regions contract into a much smaller profile. A smaller loading sheath is made possible accordingly.

In some examples, neither the proximal hoop 1506 nor the distal hoop 1508 is an “offset” hoop. In some examples, the proximal hoop 1506 is an offset hoop and the distal hoop 1508 is not offset (i.e., is a non-offset hoop). In some examples, the proximal hoop 1506 is a non-offset hoop and the distal hoop 1508 is an offset hoop. In some examples, both the proximal hoop 1506 and the distal hoop 1508 are offset hoops.

In further profile reduction aspects, certain portions of hoop and/or actuation wire can be omitted by an offset hoop. To this end, in some examples proximal and distal passage points (these may also be termed entry and exit points, or vice versa) of the hoop wire forming a hoop are offset along the length of the EPD shaft 1504. For example, a proximal passage point 1510 (or entry point of the proximal hoop wire) of the proximal hoop 1506 is offset along the EPD shaft 1504 from the distal passage point 1512 (or exit point of the proximal hoop wire). The proximal region 1522 of single layer polymer mesh is formed between these two points, or at partially bounded by a line between these two points. Similarly, in relation to the distal hoop 1508, a proximal passage point 1526 (or entry point of the distal hoop wire) is offset along the EPD shaft 1504 from a distal passage point 1528 (or exit point of the distal hoop wire). The distal passage point 1528 may be defined by an open end of the EPD shaft 1504. The distal region 1524 of single layer polymer mesh is formed between these two points, or at partially bounded by a line between these two points.

Internally of the EPD shaft 1504, the respective hoop wires may be connected to a connector (such as a connector 346 of FIG. 3) or formed integrally with an actuation wire (such as the single actuation wire 818 of FIG. 8). A single actuation wire only is needed internally of the EPD shaft 1504 in the regions between the proximal passage point 1510 and the distal passage point 1512 of the proximal hoop 1506, and the proximal passage point 1526 and the distal passage point 1528 of the distal hoop 1508. Dimensions of the EPD shaft 1504 that may need to accommodate such actuation wires can be reduced, accordingly.

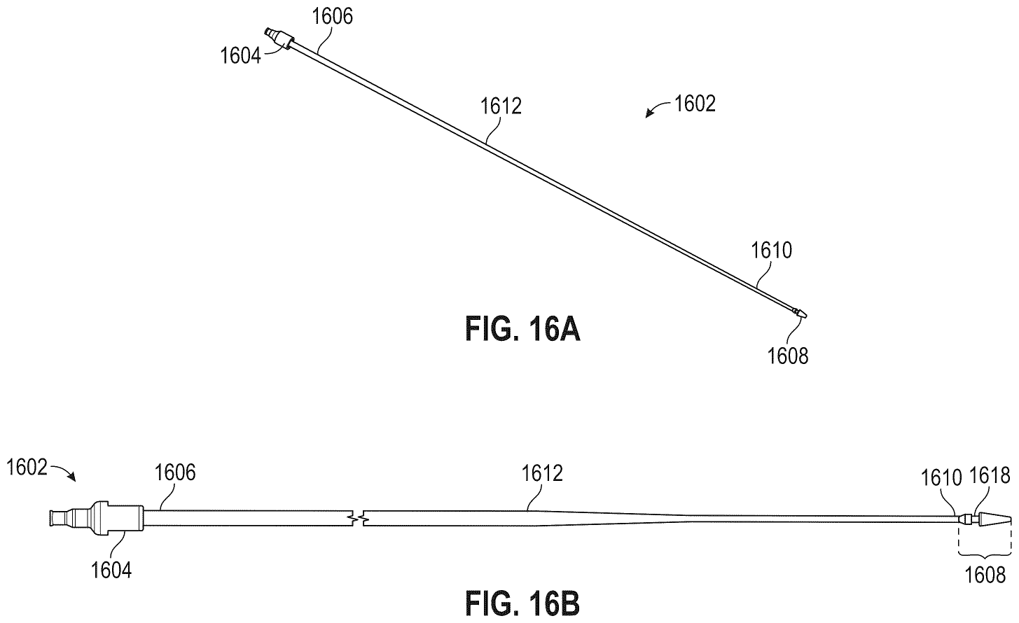

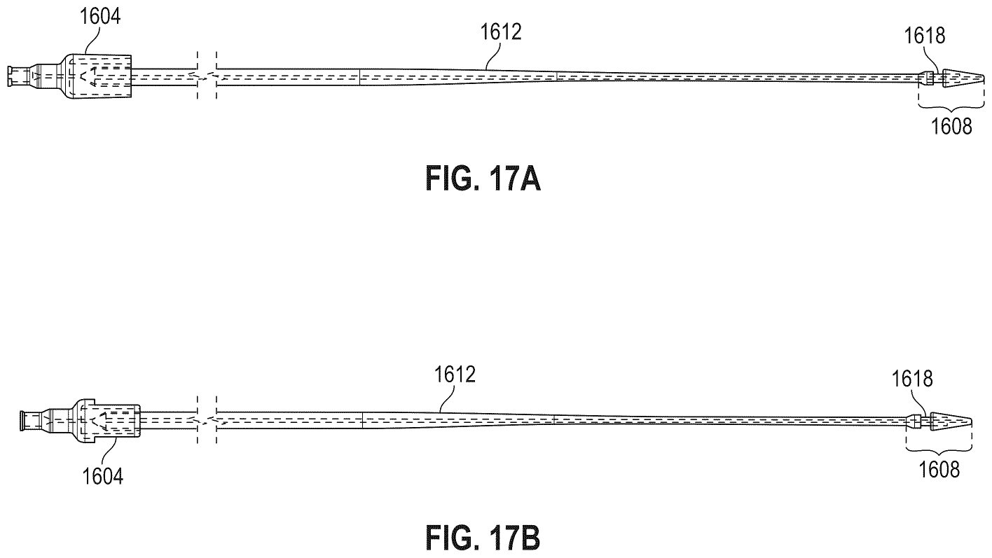

FIG. 16A and FIG. 16B show pictorial views of an example filter dilator 1602, with some of parts of the filter dilator 1602 shown in enlarged view in FIG. 16B. Sectional views of the filter dilator 1602 are provided in FIG. 17A and FIG. 17B. In FIG. 16A and FIG. 16B, the example filter dilator 1602 includes a Luer fitting 1604 provided at a proximal end 1606 of the filter dilator 1602, and a filter dilator tip 1608 provided at a distal end 1610 of the filter dilator 1602. The filter dilator tip 1608 includes a circumferential channel 1618. The filter dilator 1602 includes a flexible shaft 1612 comprised of one or more polymers (e.g., polyethylene, polypropylene, ABS, PVC, FEP, combinations thereof) or a flexible metal material or thin rod.

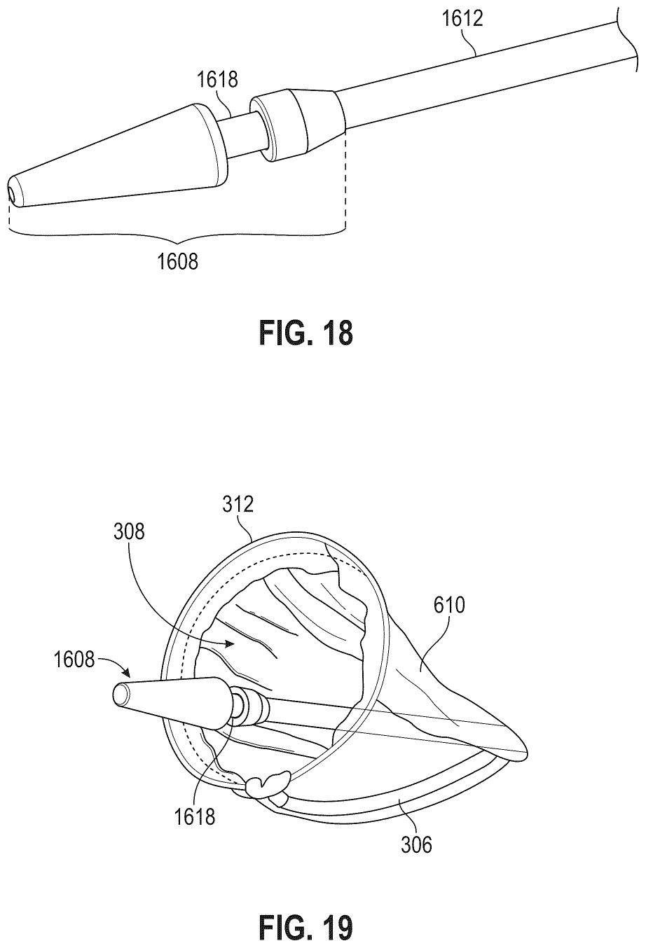

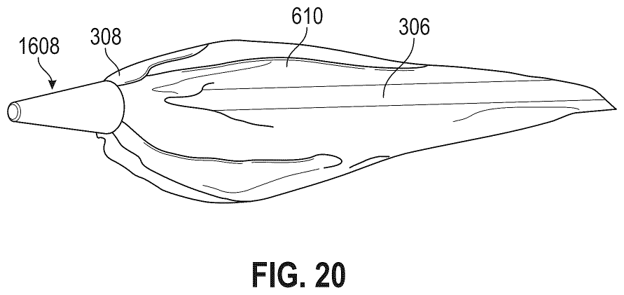

With reference to FIG. 18, the filter dilator tip 1608 of the filter dilator 1602 is coupled to the flexible shaft 1612 and includes a circumferential channel 1618 (or other formation) that can engage with and retain a distal hoop wire when the filter is closed. Pictorial views of open and closed configurations of the filter 1304 are shown in FIG. 19 and FIG. 20, respectively.

With reference back to FIG. 3, it was seen that examples of an embolic protection system 302 may include a loading tool 334. In some examples, the loading tool 334 comprises a loading sheath 336 mountable around an outside of the embolic protection device 304 to assist in loading the embolic protection device 304 into a hub of an expandable introducer 348, for example, as described further below. In some examples, the loading sheath 336 is a peel-away loading sheath, for example as described below. In some examples, each of these components may be provided or used separately, or provided and used in combination, for example as part a kit.

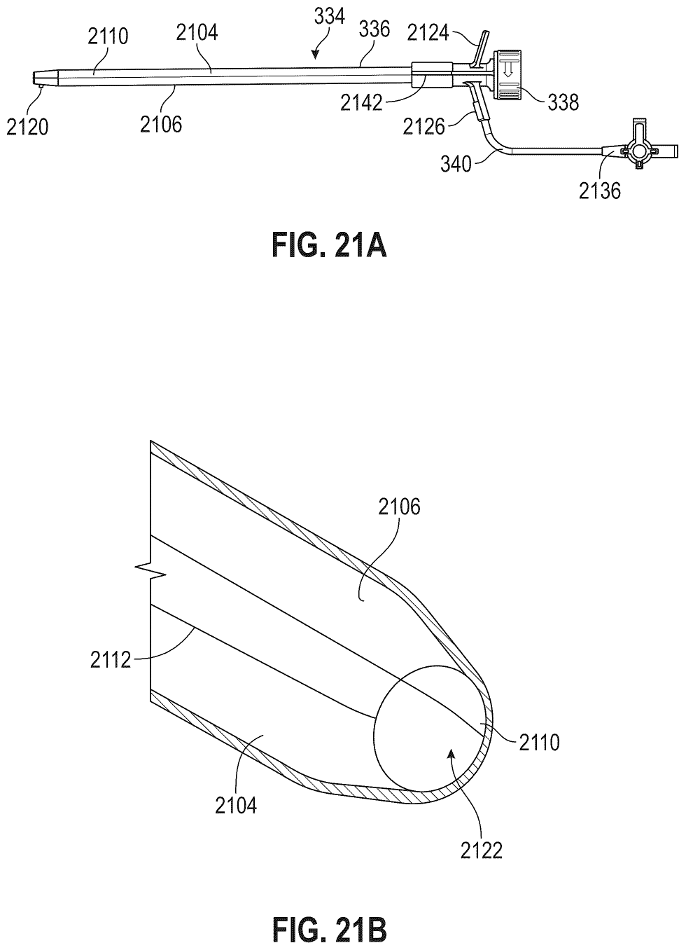

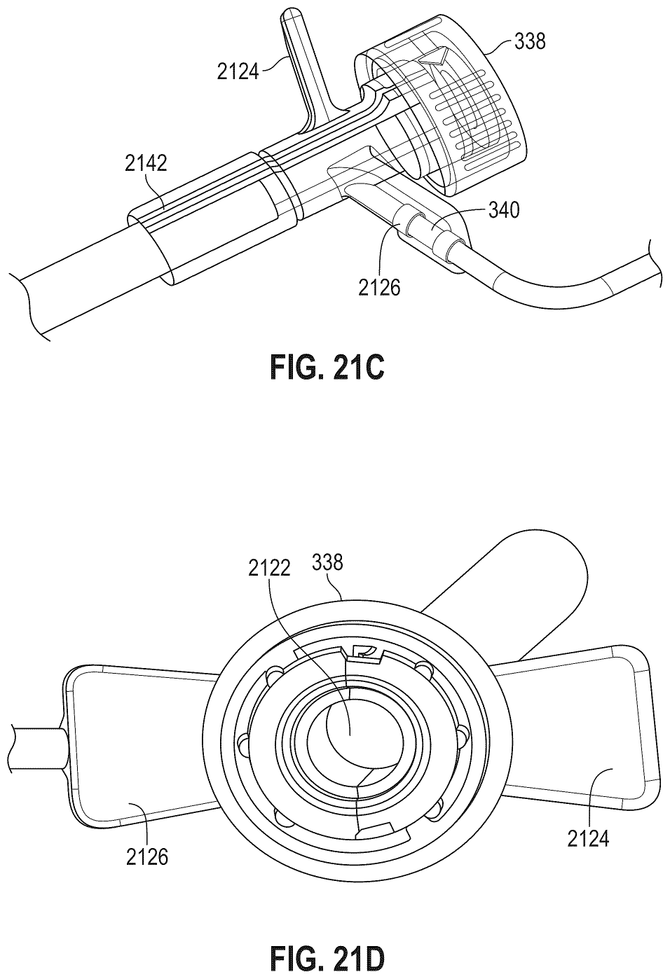

Turning now to FIG. 21A to FIG. 21D, these figures show pictorial and sectional views of the loading tool 334, according to an example embodiment. The loading tool 334 is of tubular shape defined by two separable (or peel away) parts, for example a first peel away part 2104 and a second peel away part 2106. Each peel away part is semi-circular in cross section and joined along frangible parting lines (first frangible parting line 2110 and second frangible parting line 2112) running along opposite sides of the loading sheath 336 (seen best in FIG. 21B).

The joined parts (first peel away part 2104 and second peel away part 2106) together define a tip 2120 and a lumen 2122 of the loading tool 334 into which the embolic protection system 302 can be inserted and made ready for use. The separable first peel away part 2104 and second peel away part 2106 can be split apart and peeled away from the embolic protection system 302 by manipulating two separation handles, first separation handle 2124 and second separation handle 2126. In the illustrated example of FIG. 21C, the second separation handle 2126 includes a loading tool flushing line 340 in fluid communication with the three-way stopcock 2136 and the lumen 2122 of the loading tool 334.

As shown in FIG. 21A and FIG. 21B, the first separation handle 2124 and the second separation handle 2126 are joined to each other by two parting lines 2142 that coincide with the first frangible parting line 2110 and the second frangible parting line 2112. Peeling movement of the first separation handle 2124 and the second separation handle 2126 to initiate removal of the loading tool 334 can be unlocked by rotating the loading tool hub 338 in the direction of arrow B (see FIG. 21A) that retains (when locked) the first peel away part 2104 and the second peel away part 2106 together and prevents inadvertent separation. Removal of the loading tool hub 338 from the loading tool 334 allows the first separation handle 2124 and the second separation handle 2126 to be manipulated by an operator and split apart along the two parting lines 2142, the first frangible parting line 2110, and the second frangible parting line 2112 (each of which may be perforated), thus allowing subsequent splitting apart and removal of the first peel away part 2104 and the second peel away part 2106 to release the loading sheath 336 completely from the embolic protection device 304. In some examples, the loading tool hub 338 can simply be eased off the first peel away part 2104 and the second peel away part 2106 to allow their separation. In some examples, the loading tool hub 338 includes a slit or gap in its peripheral wall allowing the loading tool hub 338 to be removed from a guide wire after release from the loading tool 334.

The embolic protection device 304 may in some examples be inserted into the human vasculature using an introducer. The introducer may be conventional or generic. An adapter for an introducer hub may be provided. In some examples, the introducer is an expandable introducer, for example an expandable introducer 348.

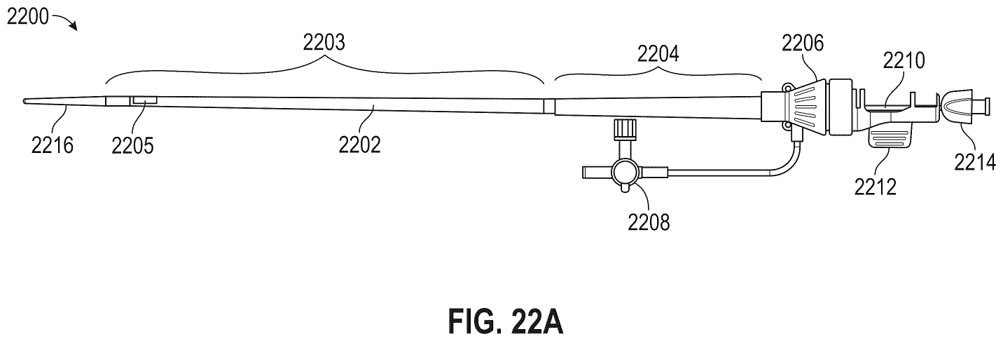

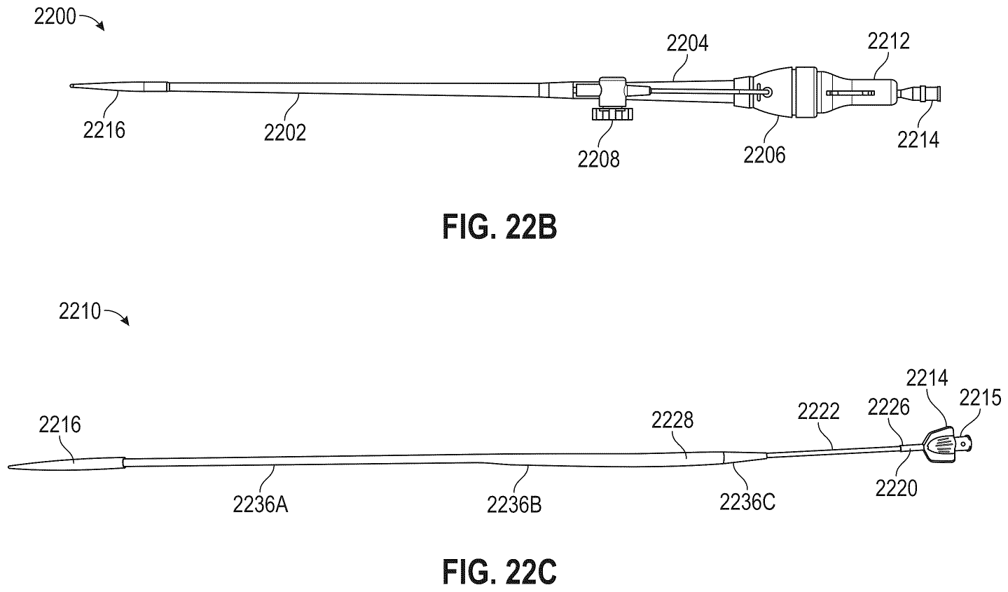

FIG. 22A through FIG. 22C show respective side and top pictorial views of an example expandable introducer 2200. The expandable introducer 2200 includes a mesh sheath 2202 (or sleeve) of expandable, porous mesh material. In some examples, the mesh material is expandable in the radial and longitudinal directions of the mesh sheath 2202. In some examples, the mesh material is expandable only in the radial direction of the mesh sheath 2202. In some examples, the mesh material is expandable only in the longitudinal direction of the mesh sheath. In some examples, the mesh sheath 2202 has a usable length of approximately 30 cm to suit an arterial dimension of the human vasculature. Some examples of this disclosure include a mesh sheath having a usable length in the range 25-35 cm to accommodate variations in vasculature size. In some examples, the mesh sheath 2202 has a contracted diameter in the range 3-7 mm and an expanded diameter in the range 7-10 mm.

In some examples, a first region of the mesh sheath 2202 is expandable and porous. For example, a first porous region 2203 may include mesh material that is expandable in the radial direction only, and may or may not be expandable axially. In some examples, the material properties of the mesh sheath may be selected so that there is no axial expansion or contraction. In other examples, the material properties may be selected so that there is some axial expansion or contraction. In some examples, as the mesh radially expands, the mesh may foreshorten 10 mm or less in the axial direction. Other arrangements are possible. In some examples, the mesh material of the first porous region 2203 includes open pores through which fluids may pass (such as blood) while embolic material such as plaque and blood clots are prevented from passing through the mesh sheath 2202.

In other examples, the mesh may not be utilized to capture embolic material but will still allow blood to pass through the membrane so that blood flow is not disrupted. A suitable mesh material for the first porous region 2203 may include polyester, nylon, or nitinol mesh. Pore sizes may be provided in the range 70-300 microns to allow blood to pass through the pores while capturing emboli or other particulates. At a distal end of the first porous region 2203 of the mesh sheath 2202, a marker 2205 may be provided. The marker 2205 may be radiopaque, echogenic, or visible under other imaging techniques known in the art. The marker 2205 may facilitate positioning of the expandable introducer in use.

In some examples, a second region of the mesh sheath 2202 is non-expandable and non-porous. In some examples, the second region may include a non-porous elastomer seal material. For examples, a second non-porous region 2204 may include a continuation of the mesh material of the first porous region 2203, but the presence of the elastomer seal material renders the second non-porous region 2204 as non-porous and it may be expandable or non-expandable. In an example, the second non-porous region 2204 may be expandable but less expandable than the first region where the mesh is disposed. A second non-porous region 2204 of the mesh sheath 2202 does not allow the passage of fluid or embolic material through the walls of the mesh sheath 2202. In some examples, the second non-porous region 2204 of the mesh sheath 2202 has a length of approximately 11 cm. Other lengths are possible to suit different applications and sizes of human vasculature.

Relative to the first porous region 2203, the second non-porous region 2204 of the mesh sheath 2202 may be held in or assume an expanded or partly expanded configuration of the mesh material, as shown. The first porous region 2203 and the second non-porous region 2204 of the mesh sheath 2202 may taper down in a distal direction along their lengths as shown to facilitate advancement of the expandable introducer 2200 into the human vasculature.

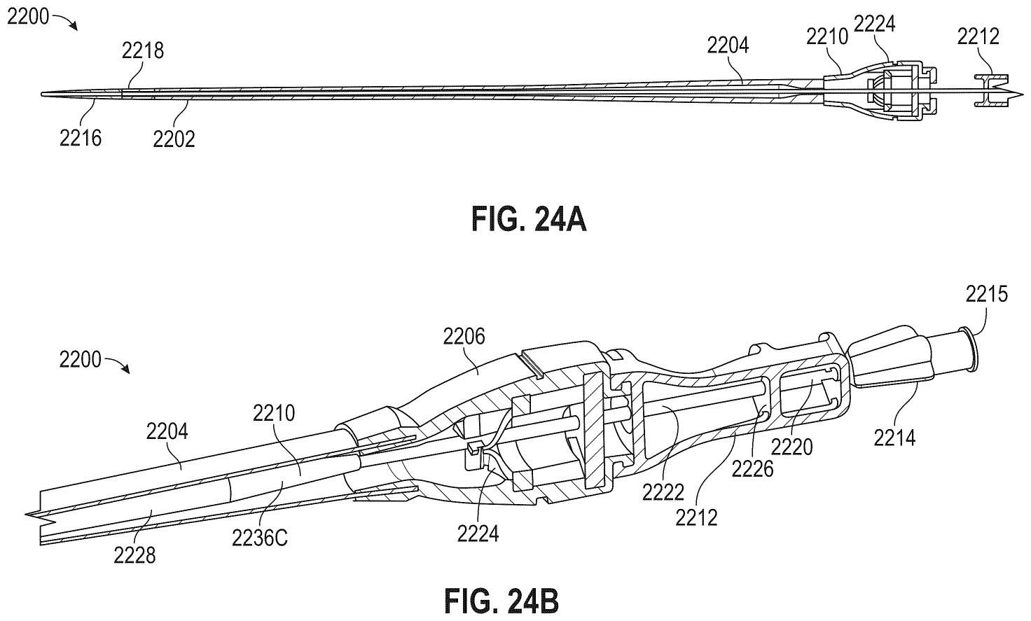

With reference again to FIG. 22A and FIG. 22B, the expandable introducer 2200 further comprises an introducer hub 2206 that includes a hemostasis valve 2224 (visible in FIG. 24A and FIG. 24B). The introducer hub 2206 is connected to a flushing three-way stopcock 2208. An interior volume of the introducer hub 2206 inside the seal of the hemostasis valve 2224 and including the mesh sheath 2202 (including the first porous region 2203 and the second non-porous region 2204) may be flushed using the flushing three-way stopcock 2208.

With reference to FIG. 22C, the expandable introducer 2200 further comprises a sheath dilator 2210 for obturating the mesh sheath 2202 or providing a removable dilating tip for insertion into the body. The sheath dilator 2210 can be locked in place within the mesh sheath 2202 of the expandable introducer 2200 by a manually removable clip 2212 (visible in FIG. 22B). The manually removable clip 2212 engages with the introducer hub 2206 and can be removed by an operator to allow the sheath dilator 2210 to advance or retract through the expandable introducer 2200 in use.

At a proximal end of the sheath dilator 2210, a dilator Luer fitting 2214 is provided. The dilator Luer fitting 2214 can be manipulated by an operator to advance or retract the sheath dilator 2210 once the manually removable clip 2212 is released, as well as allowing releasable fluid coupling with additional tubing, syringes, and/or pumps, and so forth. At an opposite distal end of the sheath dilator 2210, a sheath dilator tip 2216 is provided. The sheath dilator tip 2216 is soft and tapered to facilitate entry into the body and also to provide an atraumatic tip during distal advancement through the vessel.

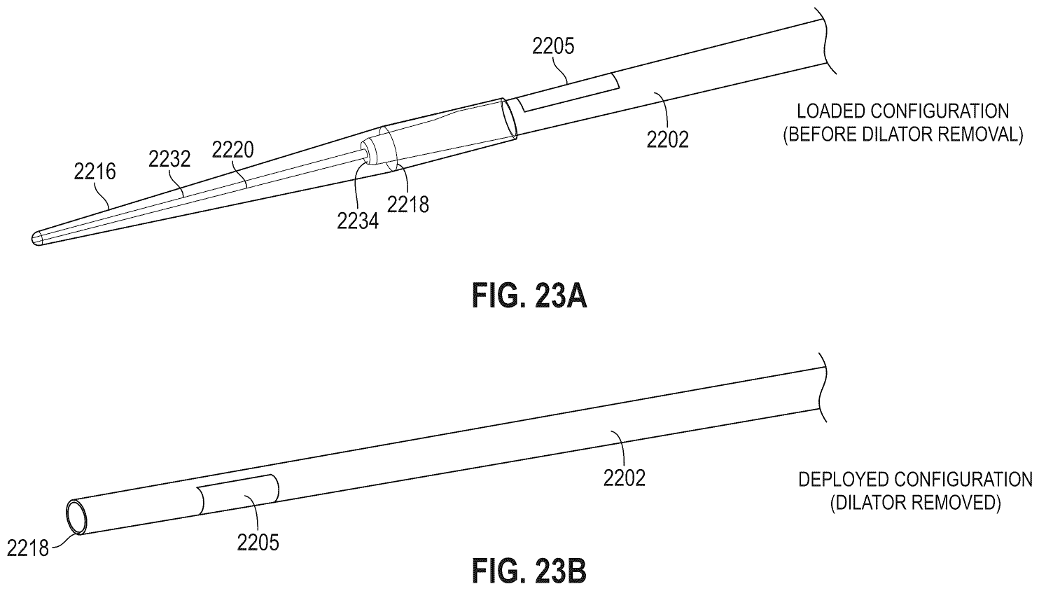

In a loaded configuration of the sheath dilator 2210, seen more clearly in FIG. 23A, the sheath dilator tip 2216 extends past and sits as a cap over a (thus-constrained) open end 2218 of the mesh sheath 2202. As described more fully below, the expandable introducer 2200 may be advanced into the human vasculature in the loaded configuration and then deployed once in position.

In a deployed configuration of the expandable introducer 2200, seen more clearly in FIG. 23B, the sheath dilator 2210 has been advanced through the mesh sheath 2202 to expand the mesh sheath 2202 and push the sheath dilator tip 2216 off the (constrained) open end 2218 of the mesh sheath 2202. This release of the sheath dilator tip 2216 allows the (now-uncapped) open end 2218 of the mesh sheath 2202 to enlarge in size. In some examples, the enlargement occurs or is driven by virtue of a natural or inherent resiliency in the material of the mesh sheath. The enlarged open end 2218 of the mesh sheath 2202 is larger than the outer diameter of the sheath dilator tip 2216. As a result, the sheath dilator 2210 is free to be retracted by an operator to withdraw the sheath dilator tip 2216 back through the enlarged open end 2218 of the mesh sheath 2202 and extract the sheath dilator 2210 and the sheath dilator tip 2216 from the expandable introducer 400 as a whole, leaving the sheath open and expanded. The introducer hub 2206 of the expandable introducer 2200 is now ready to accept introduction of the loading tool 334 and the embolic protection device 304. In some examples, the expandable introducer 2200 may be a non-expandable introducer, for example as described elsewhere herein.

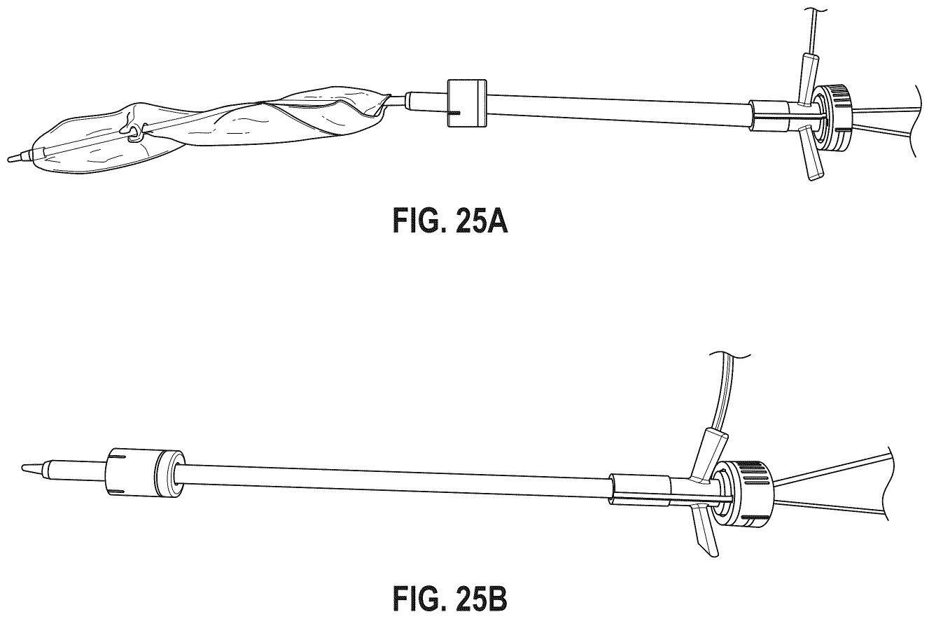

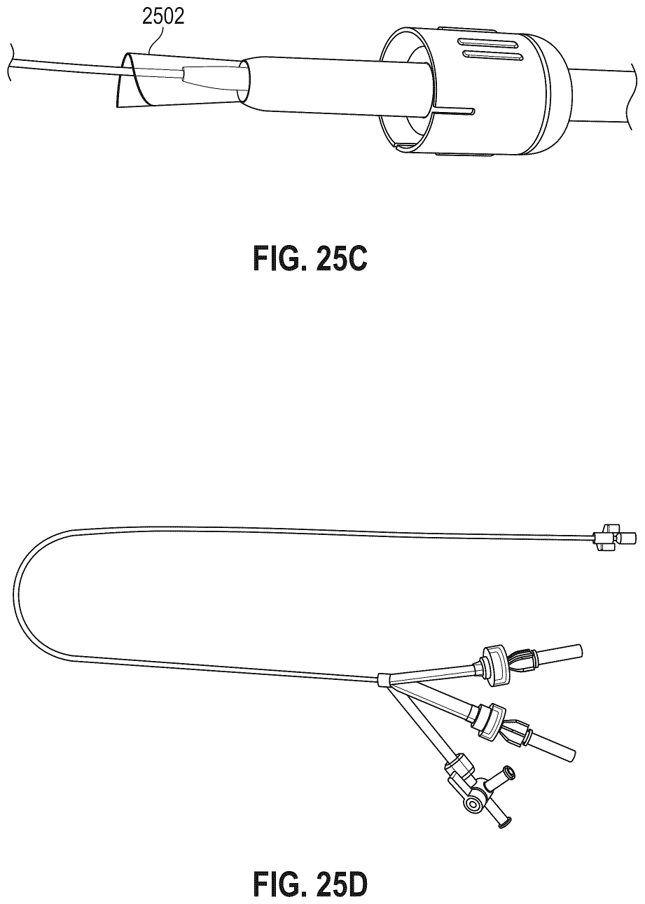

FIG. 25A through FIG. 25D show various aspects and operations in the preparation of an embolic protection device and other embolic system components for deployment in a medical procedure, according to some examples.

In FIG. 25A, both filter hoops (e.g., proximal hoop 310 and distal hoop 312 of FIG. 3) of a filter (for example filter 308) are closed. The distal hoop 312 is aligned and engaged with the filter dilator channel 512 in the filter dilator 326.

In FIG. 25B, the loading sheath 336 of a loading tool 334 is then slid over the filter 308. The loading sheath 336 is then flushed to remove air.

In FIG. 25C, a plug 2502 may be inserted into the distal end of the loading sheath 336 after flushing to keep air from re-entering. The plug 2502 is removed right before the embolic protection device 304 is inserted into an expandable introducer 348.

In FIG. 25D, the filter dilator 326 and EPD shaft 306 are also flushed to de-air.

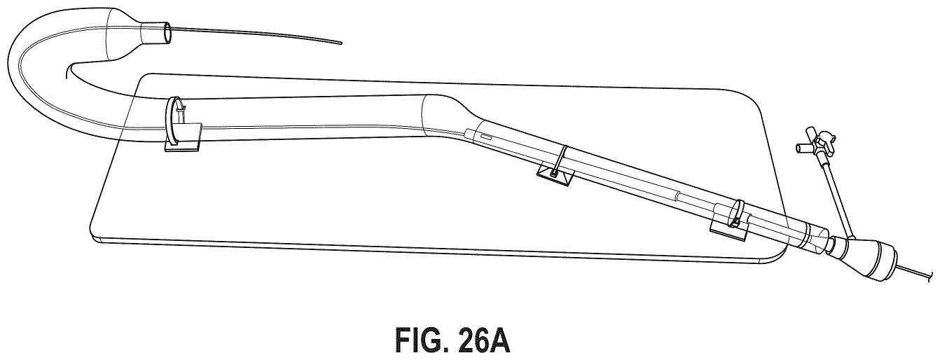

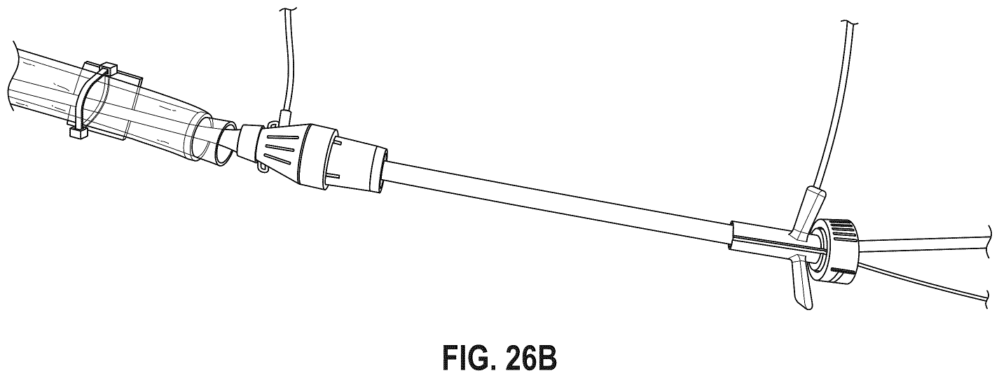

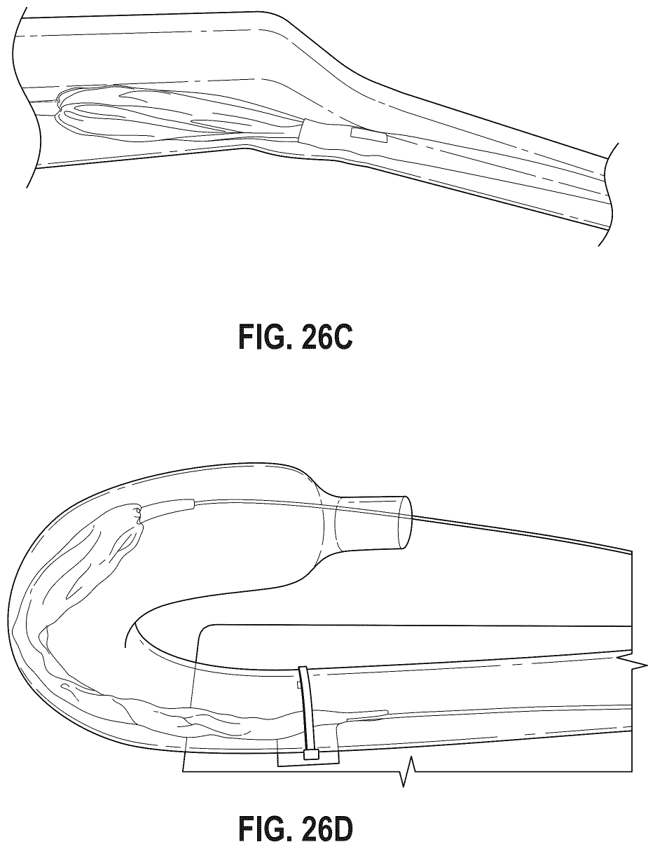

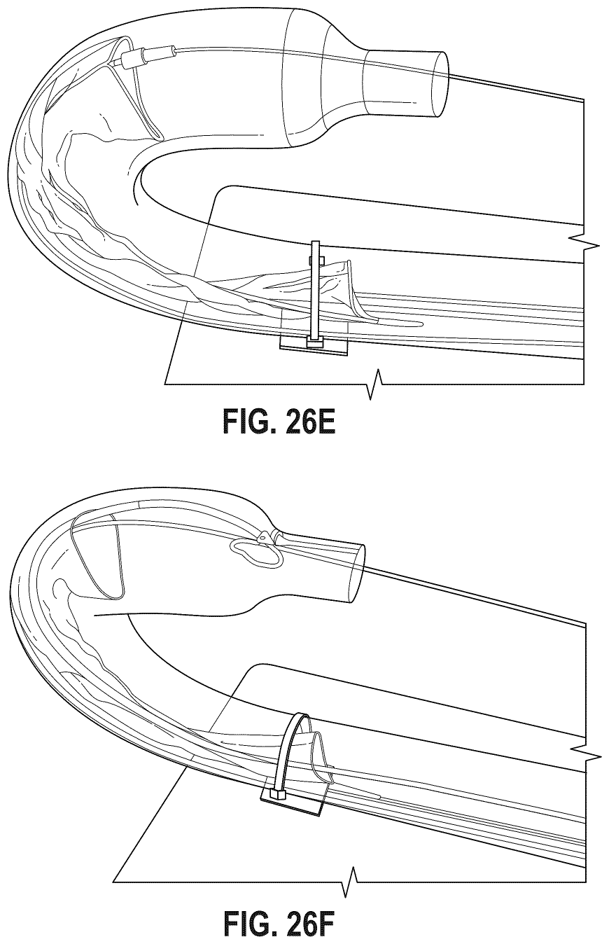

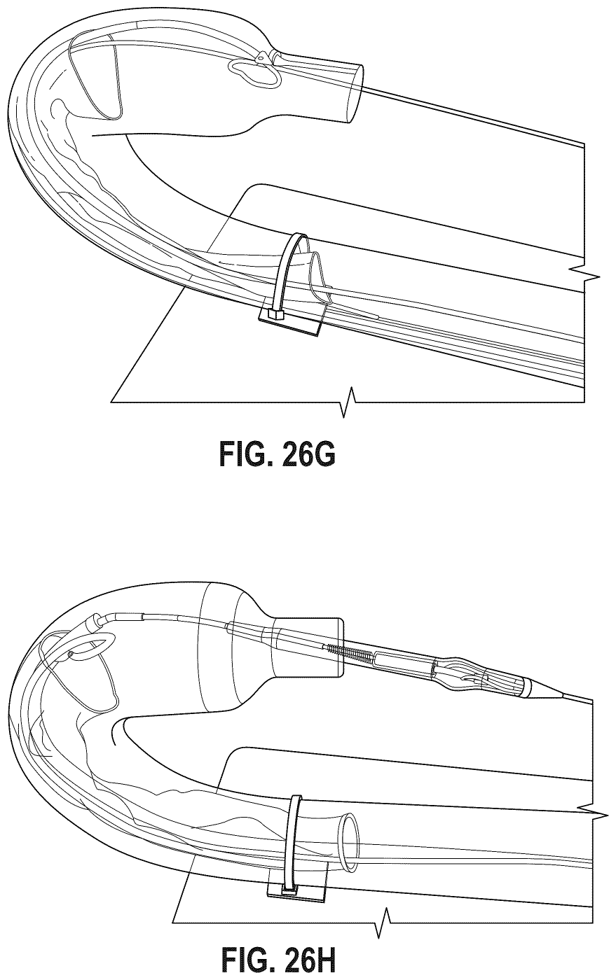

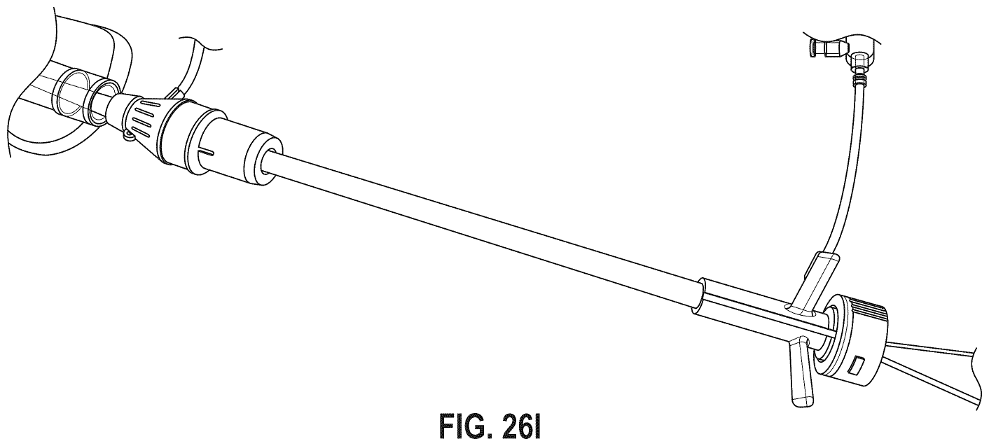

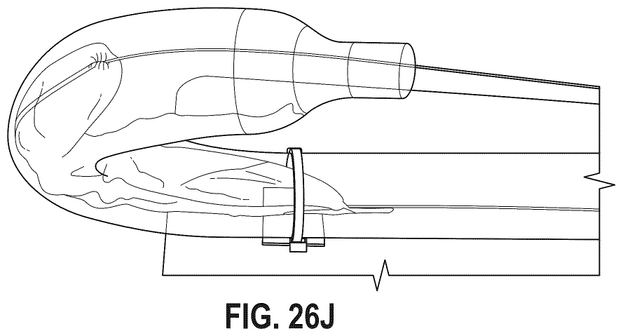

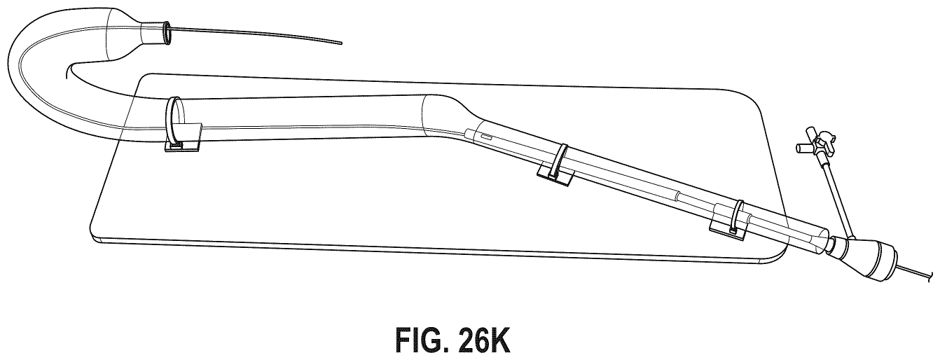

FIG. 26A through FIG. 26K show various aspects and operations in the deployment of an embolic protection device and other embolic system components for use in a medical procedure, according to example embodiments. Aspects of the human vasculature such as a femoral artery and an aorta are shown in representative form.

In FIG. 26A, the expandable introducer 348 is inserted into the femoral artery, for example a right iliac artery 930 or left iliac artery 926 of FIG. 9.

In FIG. 26B, the loading sheath 336 is inserted and locked into the expandable introducer hub.

In FIG. 26C, the embolic protection device 304 is advanced through the expandable introducer 348.

In FIG. 26D, advancement of the embolic protection device 304 continues until the filter 308 is in the ascending aorta artery beyond the brachiocephalic artery.

In FIG. 26E, the proximal hoop 310 is opened in the descending aorta and the distal hoop 312 is opened in the ascending aorta using the actuation mechanism (for example the proximal slider 622 and the distal slider 624 of FIG. 6) in the EPD hub. Both hoop diameters are adjustable to the diameter of the ascending and descending aorta. The operator (for example a doctor or surgery nurse) actuates (opens) the hoops until they can visualize each hoop conforming to the wall of the artery using fluoroscopy.

In FIG. 26F, the filter dilator 326 is removed over the guidewire and a pigtail catheter is introduced over the same guidewire, through the filter 308.

In FIG. 26G, a guidewire is then inserted through the opposite femoral artery and advanced through the filter 308 into the left ventricle. In some examples, the embolic protection device 304 is inserted into the femoral artery on one leg (contralateral side) and a surgical device (for example the TAVR device 1002 or TAVR device 1224) is inserted into the femoral artery on the other leg.

In FIG. 26H, the surgical device (for example TAVR device) is then advanced over the TAVR guidewire, and a prosthetic valve (for example) can be implanted into the patient. The filter 308 captures particulate (for example embolic debris 1014) dislodged during the procedure.

In FIG. 26I, the surgical device (TAVR device) is then removed. The TAVR guidewire is then removed through the TAVR access site (femoral). A guidewire is advanced through the pigtail catheter and the pigtail catheter is removed. The distal hoop 312 and the proximal hoop 310 are then closed.

In FIG. 26J, the embolic protection device 304 is then removed, over the guidewire, until the filter 308 is contained within the loading sheath 336 of the loading tool 334. The embolic protection device 304 is removed until the white marker on the EPD shaft 306 is visible outside of the loading tool hub 338. The guidewire remains in the artery.

In FIG. 26K, the expandable introducer 348 is left in place with the guidewire. At the end of the procedure, the expandable introducer 348 will be removed and the femoral artery (access site) is closed.

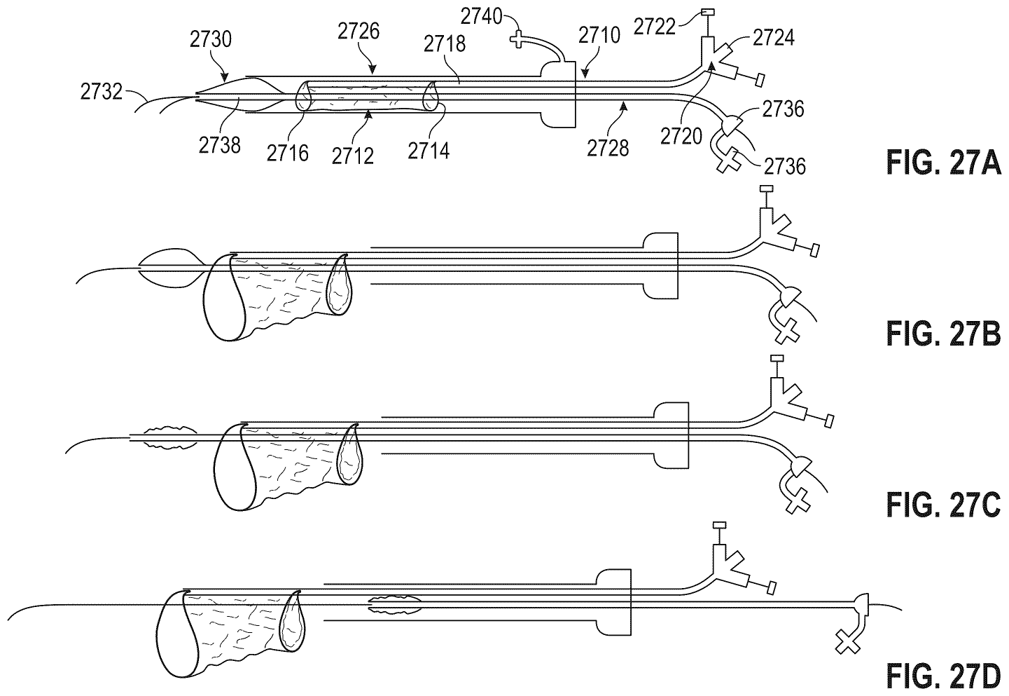

FIGS. 27A-27D show various aspects and operations in the deployment of an embolic protection device in a medical procedure, according to an example embodiment. An example embolic protection device 2710 includes an EPD shaft 2718 that supports a filter 2712. The filter 2712 includes a proximal hoop 2714 and a distal hoop 2716. The proximal hoop 2714 and the distal hoop 2716 can be controlled by actuation wires (not shown in intertest of clarity) for example as used in any one of the examples described further above. The embolic protection device 2710 includes an EPD hub 2720 that includes one or more hoop actuators 2722 and a flushing port 2724 as shown.

The example embolic protection device 2710 is shown ready for insertion into a human vasculature fitted inside an introducer sheath 2726. The introducer sheath 2726 includes an introducer sheath flushing port 2740. The filter 2712 of the embolic protection device 2710 is held in a low profile configuration inside the introducer sheath 2726. In some examples, the filter 2712 is caused to assume a closed configuration by a tightening or closing of the proximal hoop 2714 and the distal hoop 2716 using the one or more hoop actuators 2722, for example.

A dilator 2728 supports the embolic protection device 2710 and/or the filter 2712 in the deployment configuration. The dilator 2728 includes a balloon dilator tip 2730 and a dilator hub 2734. The dilator hub 2734 includes a balloon inflation/deflation port 2736 for controlling inflation (expansion) and deflation (retraction) of the balloon dilator tip 2730. The dilator 2728 is passed over a guidewire 2732 that extends through a dilator lumen 2738 of the dilator 2728 as shown. In the deployment configuration, the balloon dilator tip 2730 is inflated to conform to the inner diameter of the introducer sheath 2726. In some examples, the balloon dilator tip 2730 internally blocks the open end of the introducer sheath 2726 but does not cover the open end. The balloon dilator tip 2730 nevertheless helps to provide a matching, smooth, atraumatic supported profile for the leading end of the introducer sheath 2726 as the introducer sheath 2726 is introduced into the human vasculature.

In FIG. 27B, the dilator 2728 and the embolic protection device 2710 are advanced out of the introducer sheath 2726 after insertion of the introducer sheath 2726 into a patient's vasculature. The filter 2712 is then opened to capture embolic material during a surgical procedure by deploying one or both of the proximal hoop 2714 and the distal hoop 2716 as described further above. In FIG. 27C, the balloon dilator tip 2730 is deflated for withdrawal, as shown schematically. In FIG. 27D, the dilator 2728 with the deflated balloon dilator tip 2730 can then be removed from the introducer sheath 2726 over the guidewire 2732.

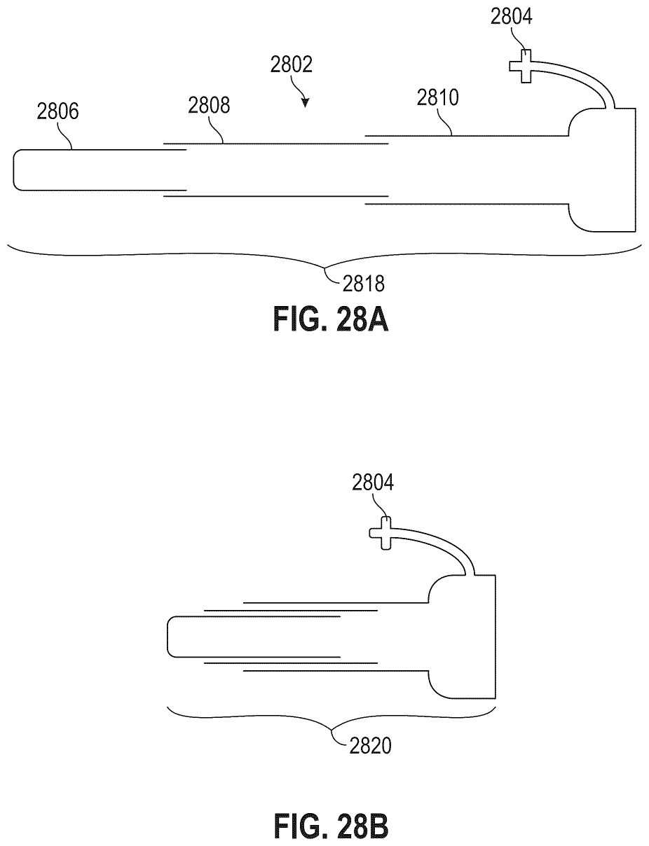

FIGS. 28A-28B show schematic views of an example telescoping introducer sheath 2802 for an embolic protection system. The telescoping introducer sheath 2802 may (or may not) form part of a loading tool in some examples, for example as the loading sheath of a loading tool 334 or as part of a loading tool 1404. The telescoping introducer sheath 2802 whether used independently or as part of a loading tool can be used to insert an embolic protection device into a human vasculature, for example as described just above with reference to FIGS. 27A-27D, or as described elsewhere herein.

The telescoping introducer sheath 2802 includes an introducer sheath flushing port 2804 and one or more sheath sections (for examples tubes) that can fit slidably or telescopically inside one another to assume an extended configuration as shown for example by a first telescoping section 2806, a second telescoping section 2808, and a third telescoping section 2810 in FIG. 28A. These sections have an extended overall length 2818. In some examples, these sections can be extended manually before deployment, or in some examples before deployment or inside the human vasculature by being urged outwardly by application of a flushing fluid (and under fluid pressure) applied to the introducer sheath flushing port 2804. When extended, the overall length of the telescoping sections is long enough to contain an embolic protection device, or at least a filter of an embolic protection device during deployment thereof.

When the telescopic sections are retracted, for example as shown in FIG. 28B, the overall length of the telescoping introducer sheath 2802 can be controllably reduced. In some examples, the overall length of the telescoping introducer sheath 2802 can be reduced manually or under application of a fluidic vacuum (suction) applied to the introducer sheath flushing port 2804 to match a length of one of the individual telescoping sections (for example first telescoping section 2806, or the second telescoping section 2808, or the third telescoping section 2810). In some examples, after introduction of an embolic protection device into an aortic artery, the length of the telescoping introducer sheath 2802 can be shortened (see for example retracted overall length 2820) by retracting the telescoping sections or tubes. This shortening may be helpful for accommodating devices such as pigtail catheters inserted though the telescoping introducer sheath 2802 that may have a shorter usable length.

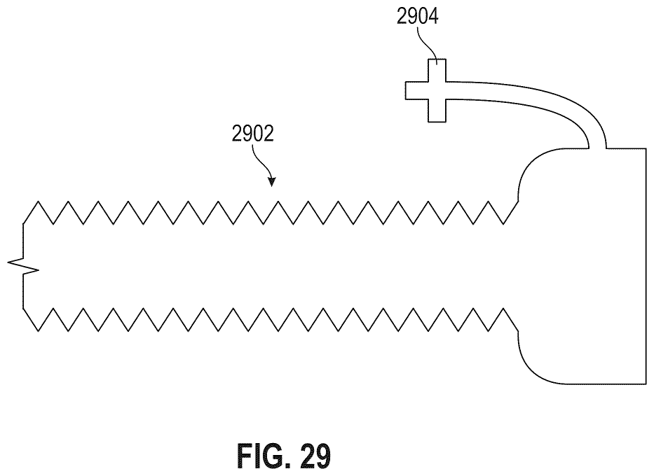

FIG. 29 shows a schematic view of an example accordion-type introducer sheath 2902 for an embolic protection system. The accordion-type introducer sheath 2902 may (or may not) form part of a loading tool in some examples, for example as the loading sheath of a loading tool 334 or as part of a loading tool 1404. The accordion-type introducer sheath 2902 whether used independently or as part of a loading tool can be used to insert an embolic protection device into a human vasculature, for example as described above with reference to FIGS. 27A-27D, or as described elsewhere herein.

The accordion-type introducer sheath 2902 includes an introducer sheath flushing port 2904 and one or more accordion-type or folding sections that can move relative to one another to control an overall length of the accordion-type introducer sheath 2902 and assume, at either extreme of movement, a fully extended or fully retracted configuration. In some examples, the accordion-type introducer sheath 2902 can be extended manually before deployment, or in some examples before deployment or inside the human vasculature by being urged outwardly by application of a flushing fluid (and under fluid pressure) applied to the introducer sheath flushing port 2904. When extended, the overall length of the accordion-type introducer sheath 2902 is long enough to contain an embolic protection device, or at least a filter of an embolic protection device during deployment thereof.

In some examples, the overall length of the accordion-type introducer sheath 2902 can be reduced manually or under application of a fluidic vacuum (suction) applied to the introducer sheath flushing port 2904 to compress the accordion-type sections or folds. The controllable reduction in length can match a length of a device such as pigtail catheter inserted though the accordion-type introducer sheath 2902 that may have a shorter usable length.

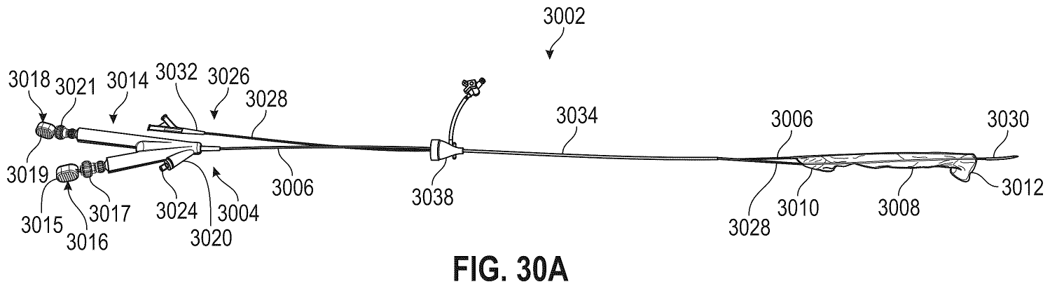

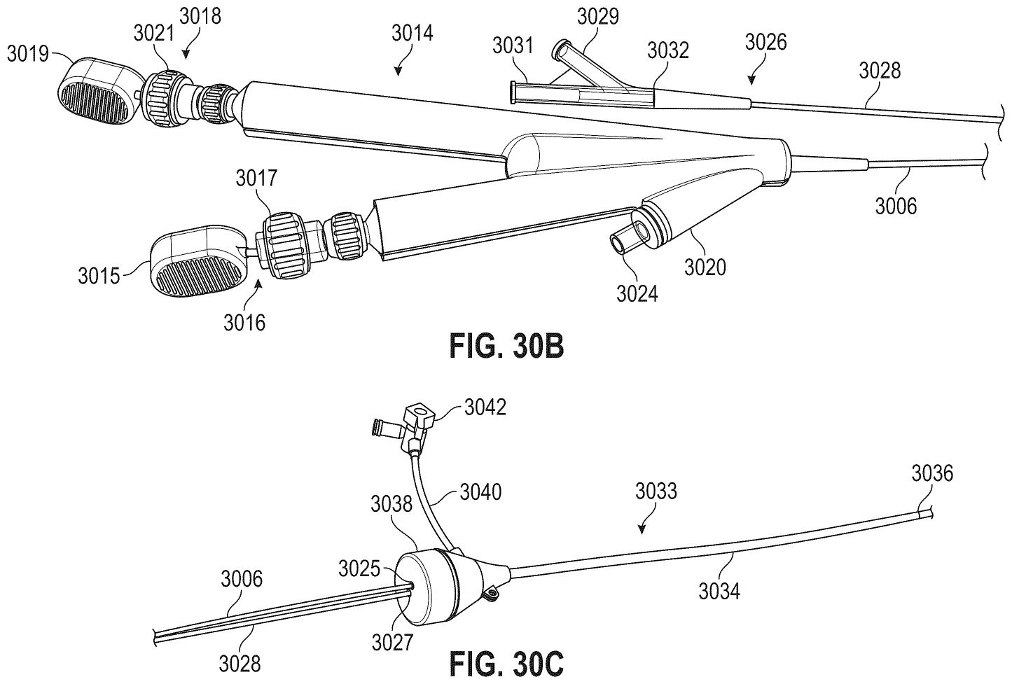

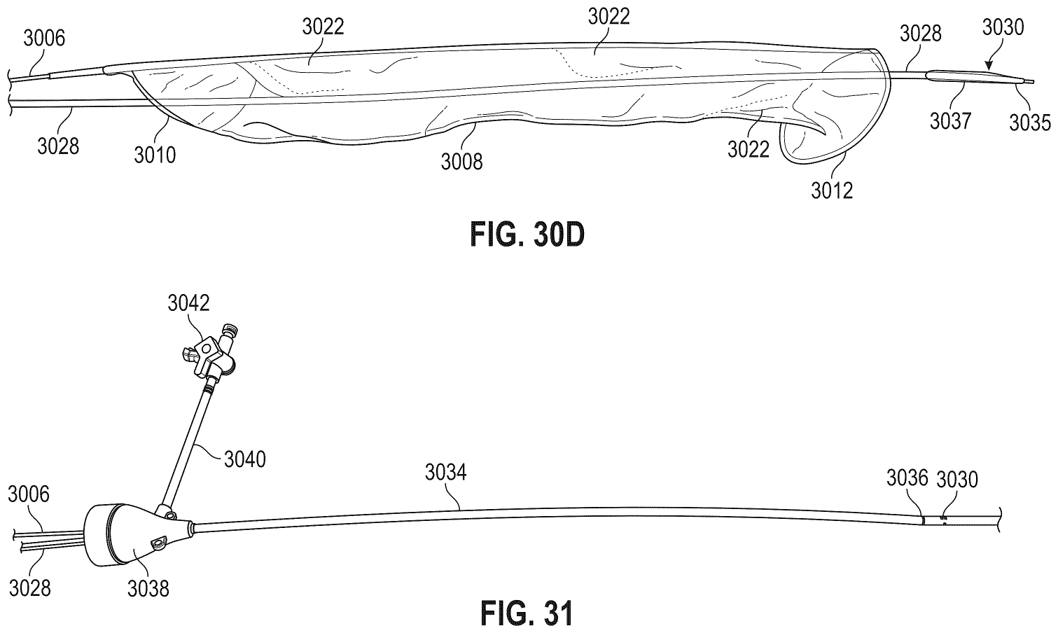

With reference to FIG. 30A-30D, examples of an embolic protection system 3002 (also known as a trans-shield system or design) may include an embolic protection device 3004 (also known as an EPD) that, viewed broadly, includes an EPD shaft 3006, a filter 3008 disposed at a distal end of the EPD shaft 3006, and an EPD hub 3014 (or handle) disposed at a proximal end of the EPD shaft 3006.

The filter 3008 is supported on a distal end of the embolic protection device 3004 and includes a proximal hoop 3010 and a distal hoop 3012 described more fully below. The filter 3008 includes a flexible mesh material to capture and retain embolic particulates and other matter. With reference to FIG. 30D, the filter 3008 may include one or more internal portions or retainers defining embolic capture pockets 3022. In some examples, for example as shown in FIG. 30A, the proximal hoop 3010 is an offset hoop while the distal hoop is a non-offset hoop. Other configurations of offset and non-offset hoops are possible, for example as described further above.

The EPD hub 3014 (or handle) includes a proximal hoop controller 3016 (or proximal actuator) and a distal hoop controller 3018 (or distal actuator). The proximal hoop controller 3016 may include a manually operable slider or push/pull knob 3015 that is connected by an internal proximal actuation wire (not visible in these views) extending through the EPD shaft 3006 to the proximal hoop 3010. The proximal hoop controller 3016 can be manipulated by an operator to control the proximal hoop 3010, for example to open or close the proximal hoop 3010 and thereby open or close a proximal end of the filter 3008.

In some examples, the proximal hoop controller 3016 includes a lockable nut 3017 that can be operated by an operator (for example by twisting) to lock the proximal hoop controller 3016 at a desired position. This desired position may in turn translate in some examples to a corresponding fully opened or closed, or partially opened or closed, configuration of the proximal hoop 3010, as desired. Other locking configurations of the proximal hoop controller 3016 are possible.

The distal hoop controller 3018 may include a manually operable slider or push/pull knob 3019 that is connected by an internal distal actuation wire (not visible in these views) extending through the EPD shaft 3006 to the distal hoop 3012. The distal hoop controller 3018 can be manipulated by an operator to control the distal hoop 3012, for example to open or close the distal hoop 3012 and thereby open or close a distal end of the filter 3008.

In some examples, the distal hoop controller 3018 includes a lockable nut 3021 that can be operated by an operator (for example by twisting) to lock the distal hoop controller 3018 at a desired position. This desired position may in turn translate in some examples to a corresponding fully opened or closed, or partially opened or closed, configuration of the distal hoop 3012, as desired. Other locking configurations of the distal hoop controller 3018 are possible.

The EPD hub 3014 further comprises an EPD flushing port 3020. The EPD flushing port 3020 may include a stopcock or valve (for example as described further above) and one or more flushing connections 3024.

In some examples, the embolic protection system 3002 further comprises a balloon dilator 3026 (also known as a filter dilator, or sheath dilator, in some examples) to assist in deploying the embolic protection device 3004 and the filter 3008. The balloon dilator 3026 includes a balloon dilator shaft 3028 (also known as a filter dilator shaft in some examples), a dilator balloon 3030 located at a distal end of the balloon dilator 3026, and a balloon dilator hub 3032 (also known as a filter dilator hub, or Luer, in some examples) located at a proximal end of the balloon dilator 3026. The balloon dilator hub 3032 includes a flushing port 3031 and a dilator inflation port 3029, as shown. The balloon dilator 3026, or more specifically the balloon dilator shaft 3028, can be advanced or retracted to pass through the proximal hoop 3010 into the filter 3008 and extend beyond the distal hoop 3012, for example as shown in FIG. 30A.

The balloon dilator 3026 can be operated to deploy the dilator balloon 3030, for example as described further below. In some examples, the balloon dilator 3026 supports or facilitates advancement of the introducer and/or advancement of the filter. In some examples, the dilator balloon 3030 includes one or more optical markers, and may include sections having a different profile or radial expansion characteristic. For example with reference to FIG. 30D, the dilator balloon 3030 may include a flat section 3037 and/or a tapered section 3035 presenting a smooth or atraumatic profile, for example as shown in that view. Other configurations of the dilator balloon 3030 are possible.

In some examples, the embolic protection system 3002 further comprises an introducer 3033. The introducer 3033 includes an introducer sheath 3034. As perhaps best seen in FIG. 30C, the EPD shaft 3006 of the embolic protection device 3004 and the balloon dilator shaft 3028 of the balloon dilator 3026 can pass through or be withdrawn from sealed apertures (for example port 3025 and port 3027) in an introducer hub 3038 of the introducer 3033 in readiness for, or during deployment of, the embolic protection system 3002 or the embolic protection device 3004. In some examples, the introducer hub 3038 includes a flushing line 3040 and a stopcock or valve 3042 to flush the introducer sheath 3034.

With reference to FIG. 31, various example aspects and operations in the preparation of an embolic protection device 3004 and embolic protection system 3002 components for use in a medical procedure are now described.

In preparation of the embolic protection system 3002 and the embolic protection device 3004, the proximal hoop 3010 and the distal hoop 3012 of the filter 3008 are closed. The proximal and distal filter hoops can be closed by operating the proximal hoop controller 3016 and the distal hoop controller 3018 as described above, for example.

The introducer sheath 3034 is then advanced over the filter 3008 and the balloon dilator 3026 until the dilator balloon 3030 is centered in a distal end 3036 (or tip) of the introducer sheath 3034, substantially as shown for example in FIG. 31.

The balloon dilator 3026 is flushed from the flushing port 3031 on the balloon dilator hub 3032, for example with heparinized saline. The EPD shaft 3006 is flushed from the EPD flushing port 3020 on the EPD hub 3014, for example with heparinized saline. The introducer sheath 3034 is flushed using the flushing line 3040 on the introducer hub 3038, for example with heparinized saline. Other flushing fluids for these flushing operations are possible.

The balloon dilator 3026 is inflated to a nominal pressure (NP) from the dilator inflation port 3029 to inflate the dilator balloon 3030. Once the dilator balloon 3030 is inflated, an airtight seal is formed to prevent air from escaping prior to insertion of the embolic protection device 3004 into the vasculature.

In some examples, the following features of the embolic protection system 3002 and embolic protection device 3004 have radiopaque markers to allow an operator to visualize the embolic protection device 3004 inside the human vasculature using fluoroscopy (x-ray).

In some examples, the distal hoop 3012 and the proximal hoop 3010 of the filter 3008 are radiopaque and they may both include an additional radiopaque marker band for enhanced visualization. In some examples, the distal end 3036, or a region immediately adjacent the distal end 3036, includes a radiopaque marker. In some examples, the balloon dilator 3026 includes two (or more) radiopaque marker bands, for example provide within the region of the dilator balloon 3030. Other materials in the embolic protection system 3002 and the embolic protection device 3004 may be visible under fluoroscopy (x-ray) by dint of being of metal or including a metallic material (for example, a coil or braid in the introducer sheath 3034 and/or the EPD shaft 3006). Other polymers may include metals (for example, tungsten or barium) extruded in the polymer to increase visualization (radiopacity).

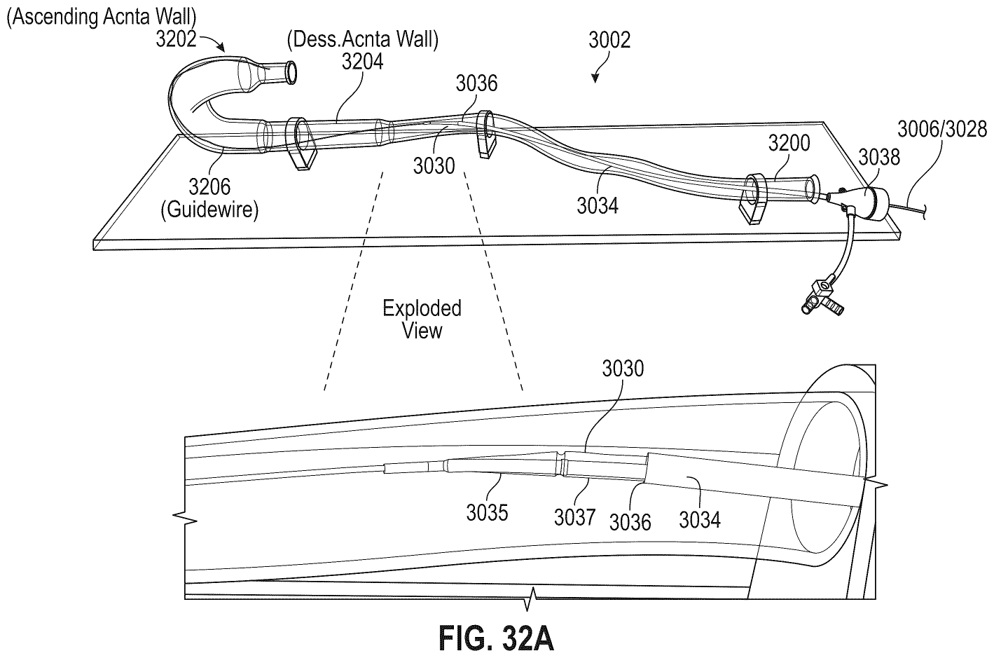

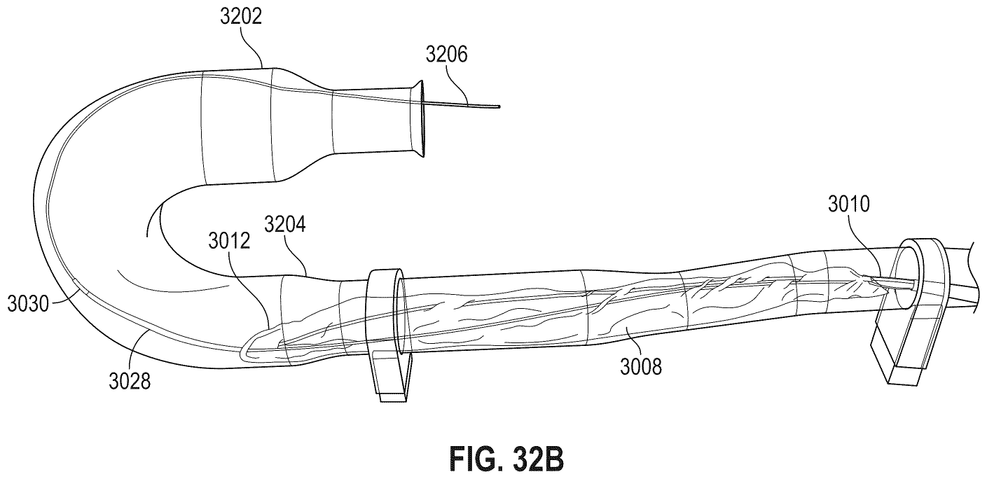

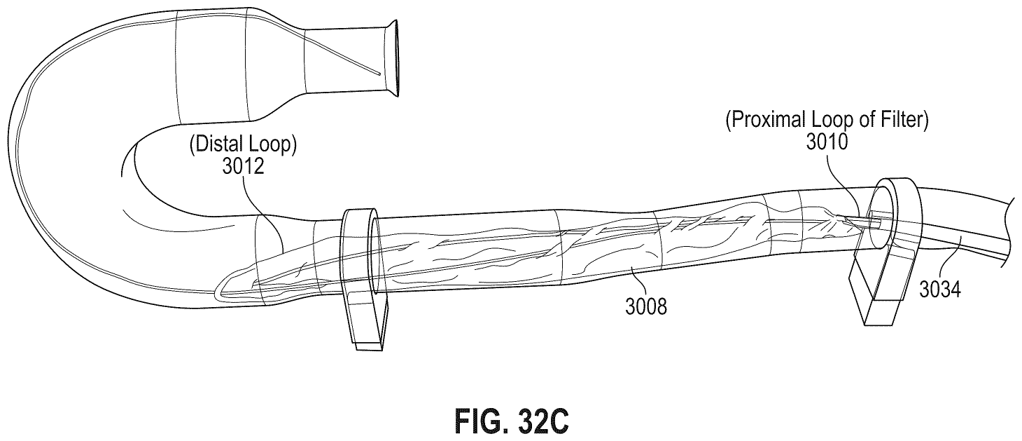

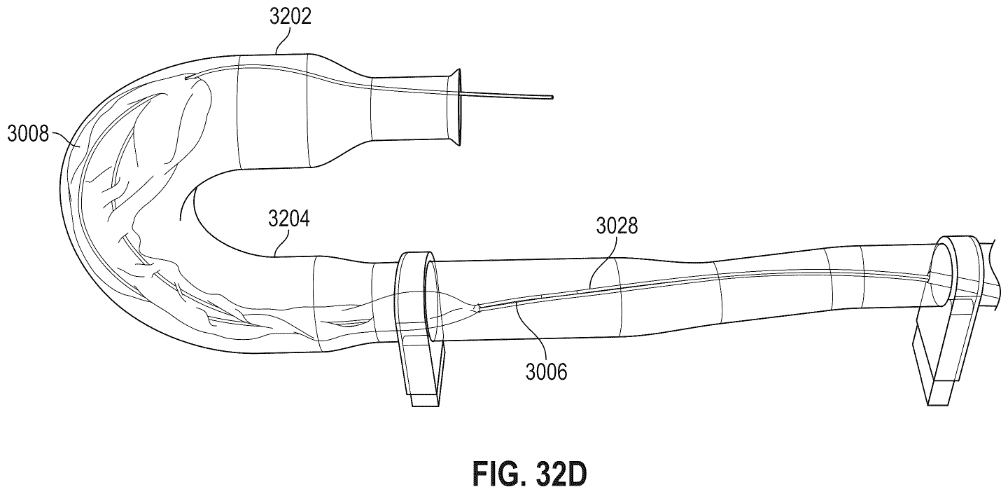

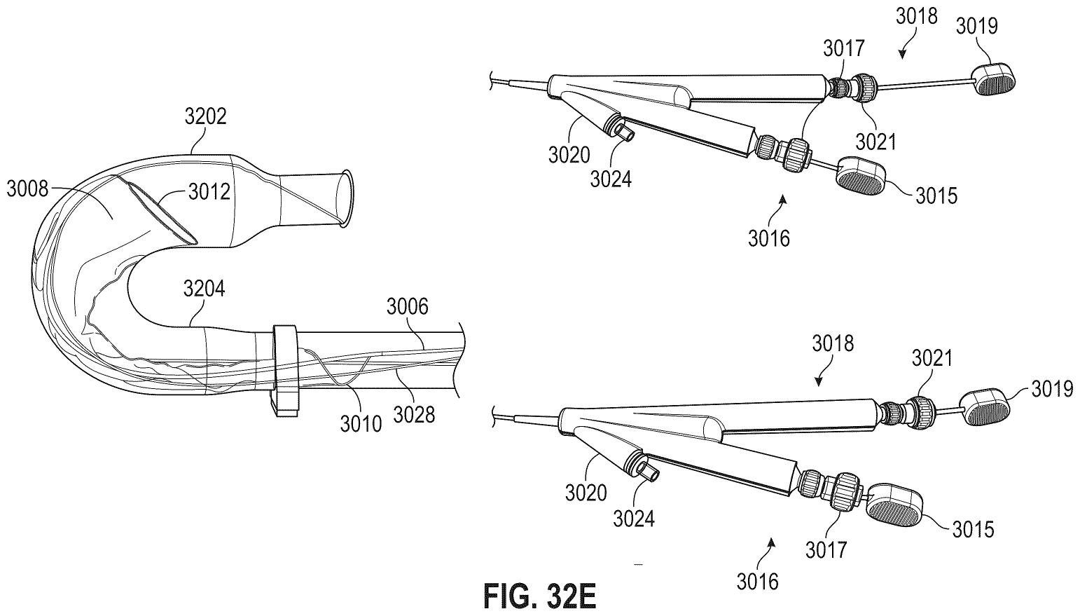

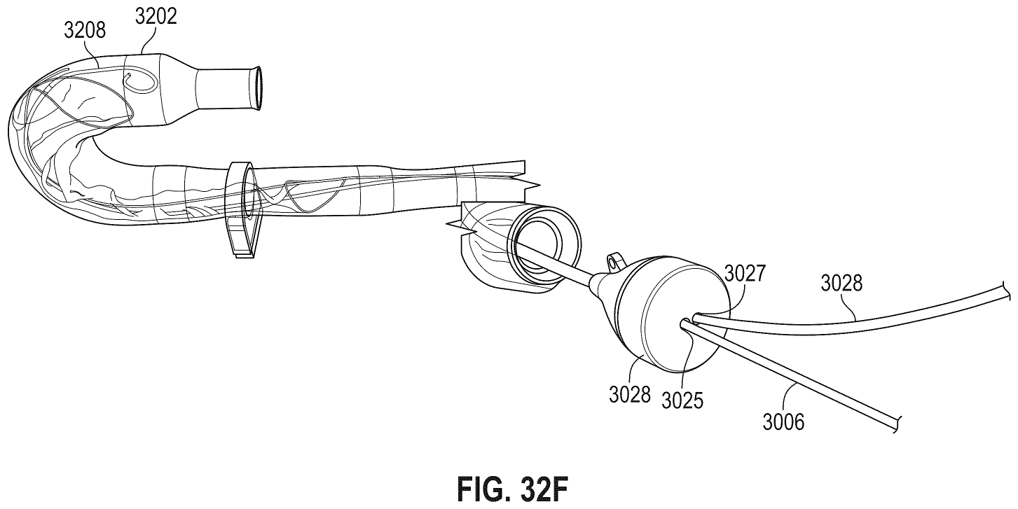

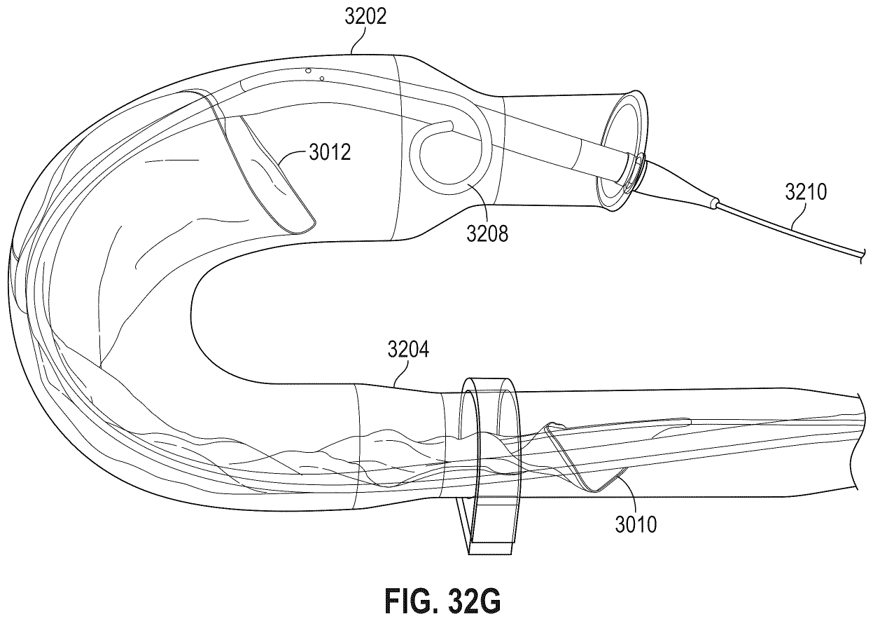

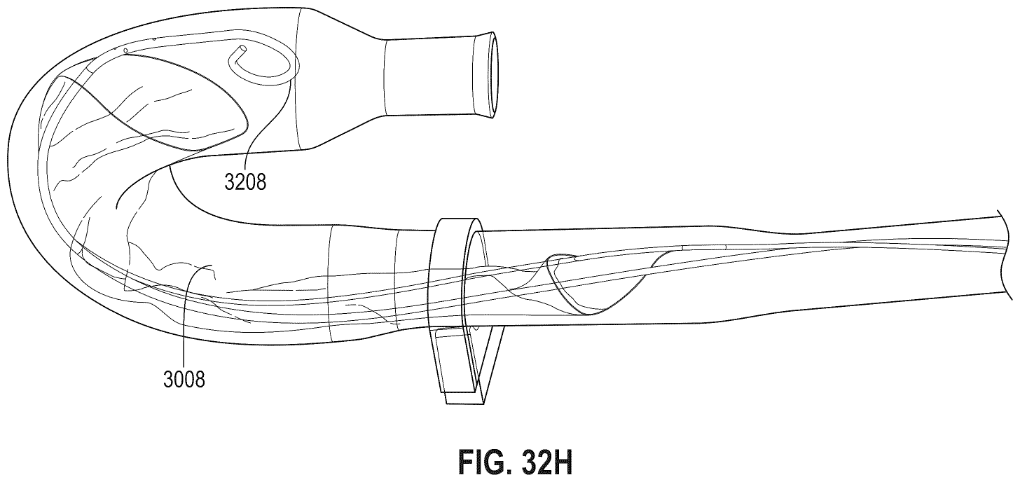

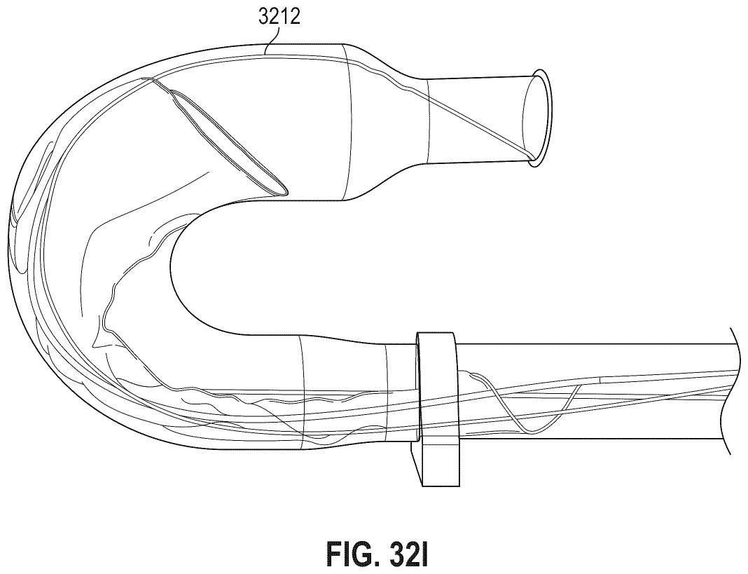

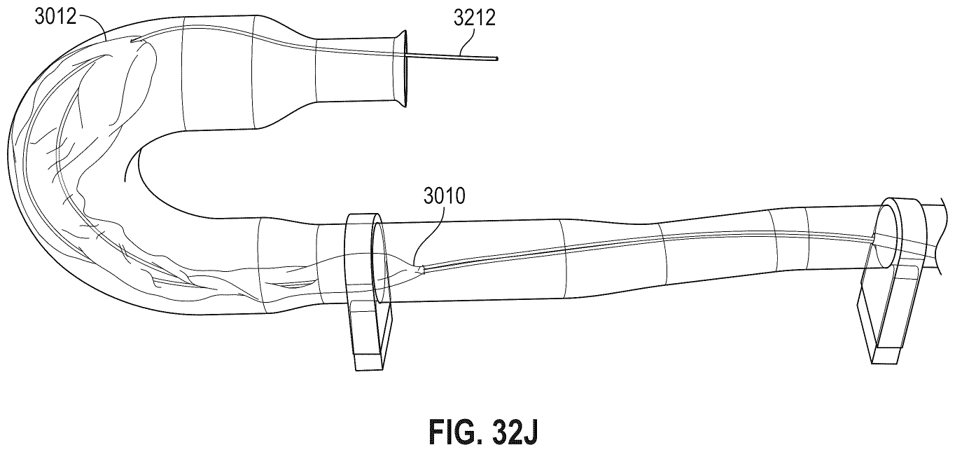

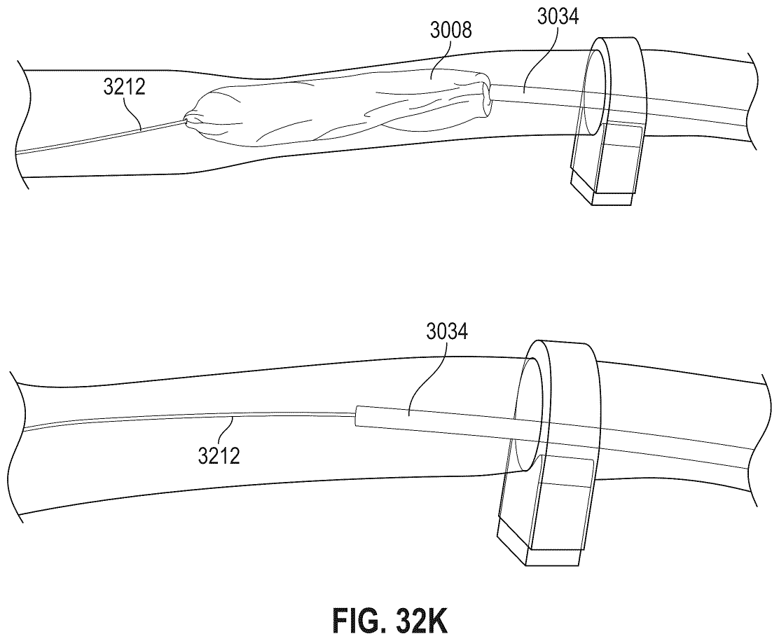

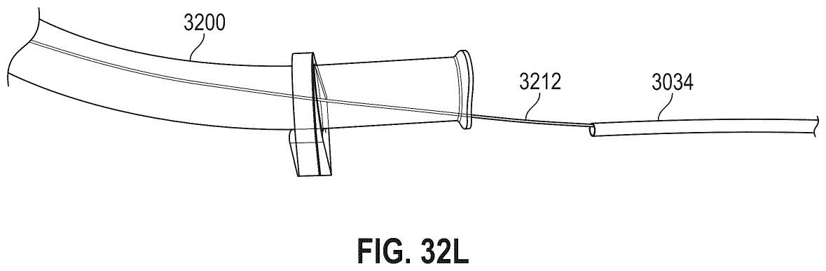

Reference is now made to FIGS. 32A-32L showing various aspects and operations in the deployment of an embolic protection device and embolic protection system in a medical procedure, according to an example embodiment.

As will now be described with reference to these views, an embolic protection system 3002 and embolic protection device 3004 is introduced into a human vasculature including a (notional, right) femoral artery 3200, but introduction of the embolic protection device 3004 into the left femoral artery is also possible. In some examples, a device such as a TAVR device (for example a TAVR device 1002 shown in FIG. 10) can be introduced into an artery opposite to that of the embolic protection device 3004. The illustrated notional human vasculature in FIG. 32A also includes an ascending aorta 3202 and a descending aorta 3204. A guidewire 3206 is shown passed through the illustrated vasculature. The left subclavian, left common carotid, and the innominate artery adjacent the ascending aorta are not shown in the interest of clarity.

In FIG. 32A, the embolic protection system 3002 as prepared above is inserted into the femoral artery 3200 until the distal end 3036 of the introducer sheath 3034 is positioned in the descending aorta 3204, for example substantially as shown. As seen in the exploded view of FIG. 32A, the dilator balloon 3030 is in an inflated configuration during advancement of the introducer sheath 3034 such that an outer diameter of the dilator balloon 3030 mates (or substantially matches) with an inner diameter of the introducer sheath 3034 for a seamless profile or transition at that juncture during insertion. As shown the dilator balloon has a tapered section 3035 for smooth, atraumatic insertion though the femoral and iliac arteries. The dilator balloon 3030 also includes a flat section 3037 of larger diameter partially inserted into the distal end 3036 of the introducer sheath 3034. Other configurations of the dilator balloon 3030 are possible.

In FIG. 32B, the dilator balloon 3030 is deflated after inserting the introducer sheath 3034 into the descending aorta 3204. The filter 3008 and the balloon dilator 3026 are then advanced out of the introducer sheath 3034 into the descending aorta 3204.

In FIG. 32C, the balloon dilator 3026 is then removed from the introducer sheath 3034 leaving the filter 3008 in the descending aorta 3204.

In FIG. 32D, the filter 3008 is then advanced around the aortic arch to the ascending aorta 3202. In one example operation, the balloon dilator 3026 has already been removed from the descending aorta 3204. In another example operation, the filter 3008 and the balloon dilator shaft 3028 are tracked to the ascending aorta 3202 before the balloon dilator 3026 is removed. In another example operation, the balloon dilator 3026 is tracked to the ascending aorta 3202 with the dilator balloon 3030 in an inflated or deflated configuration.

In another example operation, in the descending aorta 3204, the deflated dilator balloon 3030 is pulled back proximally so that it is disposed within the distal hoop 3012 of the filter 3008. The dilator balloon 3030 is then inflated to lock into the distal hoop 3012. In this configuration, when the balloon dilator 3026 and the filter 3008 are tracked over the aortic arch to the ascending aorta 3202, the dilator balloon 3030 and the filter 3008 are locked into each other.

In FIG. 32E, the distal hoop 3012 is opened in the ascending aorta 3202 and the proximal hoop 3010 is opened in the descending aorta 3204 using the corresponding distal hoop controller 3018 and proximal hoop controller 3016. Example configurations of the distal hoop controller 3018 and the proximal hoop controller 3016 are shown in the inset views of FIG. 32E. In the top inset view, the distal hoop controller 3018 (more specifically the manually operable slider or push/pull knob 3019) is pulled back to close the distal hoop 3012. In the same inset view, the proximal hoop controller 3016 (more specifically the manually operable slider or push/pull knob 3015) is pulled back to close the proximal hoop 3010. In the lower inset view, the distal hoop controller 3018 (more specifically the manually operable slider or push/pull knob 3019) has been advanced to open the distal hoop 3012. In the same inset view, the proximal hoop controller 3016 (more specifically the manually operable slider or push/pull knob 3015) has been advanced to open the proximal hoop 3010. Many other configurations of the proximal hoop controller 3016 and the distal hoop controller 3018 are possible to fully open or close, or partially open or close, the proximal hoop 3010 and the distal hoop 3012. At any of these configurations, the proximal hoop controller 3016 and the distal hoop controller 3018 may be individually locked and unlocked by rotating the lockable nut 3017 and/or the lockable nut 3021.

In FIG. 32F, a catheter (for example, a pigtail catheter 3208) is then advanced over the guidewire 3206 (typically the same guidewire that the balloon dilator 3026 was advanced over) through the filter 3008. The guidewire 3206 is removed leaving the pigtail catheter 3208 in the ascending aorta 3202. In some examples, for example as shown in the inset view of FIG. 32F, a pigtail catheter shaft may utilize the same port (for example the port 3027) of the introducer hub 3038 as was used by the balloon dilator shaft 3028 (for example as shown in FIG. 30C above).

In FIG. 32G, from the opposing femoral artery (leg), a guidewire 3210 is tracked through the filter 3008 and into the left ventricle of the heart. When tracking the guidewire 3210 through the proximal hoop 3010 (in the descending aorta 3204) there is a possibility that the guidewire 3210 tracks past the proximal hoop 3010 on the outside (i.e., between the proximal hoop 3010 and the descending aorta 3204). In order to check for this possible occurrence, a closing of the proximal hoop 3010 and consequent disturbance of the guidewire 3210 can be tracked (viewed under fluoroscopy) to confirm if the guidewire 3210 is inside the proximal hoop 3010 or outside the proximal hoop 3010.

An interventional device (for example, a transcatheter aortic valve replacement (TAVR) device or a valvuloplasty balloon catheter) is then tracked over the guidewire 3210 from the opposing femoral artery of artery use by the embolic protection device 3004. A heart procedure using the interventional device is performed with the filter 3008 open (i.e., the distal hoop 3012 open) to capture and trap embolic material. In some examples, the proximal hoop 3010 in the descending aorta 3204 is closed.

In FIG. 32H, after interventional device use is complete, the interventional device is removed over the guidewire 3210 from the vasculature. Once all interventions are complete (sometimes more than one procedure is performed), the guidewire 3210 is removed through the filter 3008 and out the opposing femoral artery vasculature. At this point, only the pigtail catheter 3208 remains through the filter 3008.

In FIG. 32I, a guidewire 3212 is inserted through the pigtail catheter 3208 and the pigtail catheter 3208 is removed.

In FIG. 32J, the distal hoop 3012 and the proximal hoop 3010 are closed.

In the views of FIG. 32K, the filter 3008 is pulled back into the introducer sheath 3034. In the top view, the filter 3008 is shown partially inside the introducer sheath 3034. In the bottom view, the filter 3008 has been fully pulled back to lie completely inside the introducer sheath 3034.

In FIG. 32L, the introducer sheath 3034 is then removed from the femoral artery 3200 leaving the guidewire 3212 in place. The guidewire 3212 is then removed from the human vasculature to complete the medical procedure. In some examples, the guidewire 3212 is removed with the introducer sheath.

In summary, as described above, the example protective configurations of the embolic protection device and embolic protection system provide a means for conducting an intervention while also protecting the underlying tissue and related anatomy.

Various exemplary embodiments of the inventive subject matter are described herein. Reference is made to these examples in a non-limiting sense. They are provided to illustrate more universally applicable aspects of the inventive subject matter. Various changes may be made to the inventive subject matter described without departing from the scope of the inventive subject matter as defined by the appended claims. In addition, many modifications may be made to adapt a particular situation, material, composition of matter, process, process act(s) or step(s) to the objective(s), or scope of the present subject matter. Further, as will be appreciated by those with skill in the art that each of the individual variations described and illustrated herein has discrete components and features which may be readily separated from or combined with the features of any of the other several embodiments without departing from the scope of the present embodiments. All such modifications are intended to be within the scope of claims that may be associated with this disclosure.

Any of the devices described for conducting the subject diagnostic or interventional procedures may be provided in packaged combination for use in executing such interventions. These supply “kits” may further include instructions for use and be packaged in sterile trays or containers as commonly employed for such purposes.

The present application discloses methods that may be performed using the subject devices. The methods may comprise the act of providing such a suitable device. Such provision may be performed by the end user. In other words, the “providing” act merely requires the end user obtain, access, approach, position, set-up, activate, power-up, or otherwise act to provide the requisite device in the subject method. Methods recited herein may be conducted in any order of the recited events that is logically possible, as well as in the recited order of events.

Exemplary aspects of the inventive subject matter, together with details regarding material selection and manufacture have been set forth above. As for other details of the present inventive subject matter, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the present disclosure in terms of additional acts as commonly or logically employed.

In addition, though the inventive subject matter has been described in reference to several examples optionally incorporating various features, the inventive subject matter is not to be limited to that which is described or indicated as contemplated with respect to each variation of the inventive subject matter. Various changes may be made to the inventive subject matter described without departing from the scope of the inventive subject matter defined by the appended claims. In addition, where a range of values is provided, it is understood that every intervening value, between the upper and lower limit of that range and any other stated or intervening value in that stated range, is encompassed within the inventive subject matter.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless specifically stated otherwise. In other words, use of the articles allows for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only,” and the like in connection with the recitation of claim elements, or use of a “negative” limitation.

Without the use of such exclusive terminology, the term “comprising” or “including” in claims associated with this disclosure shall allow for the inclusion of any additional element, irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present inventive subject matter is not to be limited to the examples provided and/or the subject specification, but rather only by the scope of claim language associated with this disclosure as defined by the appended claims.

What is claimed is:

1. An embolic protection system comprising:
an embolic protection device (EPD) including:
an EPD shaft;
a dual-hoop filter provided at a distal end of the EPD shaft, the dual-hoop filter including a distal hoop provided at a distal end of the dual-hoop filter, and a proximal hoop provided at a proximal end of the dual-hoop filter; and
a filter actuator located at or towards a proximal end of the EPD, the filter actuator operable to selectively open or close the distal hoop and the proximal hoop of the dual-hoop filter.

2. The embolic protection system of claim 1, further comprising a dilator.

3. The embolic protection system of claim 2, wherein the dilator includes an expandable tip.

4. The embolic protection system of claim 1, further comprising a loading tool.

5. The embolic protection system of claim 1, further comprising an introducer and/or an introducer sheath.

6. A method of deploying an embolic protection device (EPD) into a vasculature of a patient, the method comprising:
establishing access to the vasculature;
introducing a guide wire into the vasculature to assist with guidance of the embolic protection device through the vasculature;
advancing the embolic protection device into the vasculature and over the guide wire toward a target treatment region, wherein the embolic protection device comprises an EPD shaft, a dual-hoop filter provided at a distal end of the EPD shaft, the dual-hoop filter including a distal hoop provided at a distal end of the dual-hoop filter, and a proximal hoop provided at proximal end of the dual-hoop filter, and a filter actuator located at or towards a proximal end of the EPD, the filter actuator operable to selectively open or close the distal hoop and the proximal hoop of the dual-hoop filter;
actuating the filter actuator thereby radially expanding at least one of the distal hoop and the proximal hoop of the dual-hoop filter to lie against a vessel wall at the target treatment region; and
capturing emboli in the dual-hoop filter.

7. The method of claim 6, further comprising inserting a therapeutic device through the dual-hoop filter and advancing the therapeutic device toward the target treatment region.

8. The method of claim 6, wherein the target treatment region comprises an aortic valve.

9. The method of claim 7, further comprising, after a withdrawal of the therapeutic device through the dual-hoop filter, closing at least one of the distal hoop and the proximal hoop of the dual-hoop filter.

10. An embolic protection device (EPD) including:
an EPD shaft;
a dual-hoop filter provided at a distal end of the EPD shaft, the dual-hoop filter including a distal hoop provided at a distal end of the dual-hoop filter, and a proximal hoop provided at proximal end of the dual-hoop filter; and
a filter actuator located at or towards a proximal end of the EPD, the filter actuator operable to selectively open or close the distal hoop and the proximal hoop of the dual-hoop filter.

11. The EPD of claim 10, wherein the dual-hoop filter includes a polymer mesh.

12. The EPD of claim 10, wherein the dual-hoop filter includes a particulate entrapment feature.

13. The EPD of claim 12, wherein the particulate entrapment feature defines a passage or gap in the dual-hoop filter allowing a therapeutic device to pass therethrough when the therapeutic device is passed through the distal hoop and the proximal hoop of the dual-hoop filter.

14. The EPD of claim 10, wherein at least one of the distal hoop and the proximal hoop is formed integrally with an actuation wire extending between the dual-hoop filter and the filter actuator.

15. The EPD of claim 10, wherein the filter actuator includes a distal slider to operate the distal hoop, and a proximal slider to operate the proximal hoop.

16. The EPD of claim 10, wherein the filter actuator includes a single slider to operate the distal hoop and the proximal hoop simultaneously.

17. The EPD of claim 10, wherein at least one of the distal hoop and the proximal hoop includes nitinol material and is pre-shaped to open in a round or oval configuration to conform to an aortic wall at a target treatment region.

18. The EPD of claim 10, wherein a central portion of the dual-hoop filter, when opened, has a lower profile than both end portions of the dual-hoop filter when the distal hoop and the proximal hoop are open.

19. The EPD of claim 10, wherein the EPD shaft includes a continuous braided polymer extrusion.

20. The EPD of claim 10, wherein the EPD shaft is a composite shaft and includes a distal shaft and a proximal shaft.

21. The EPD of claim 10, wherein at least one of the distal hoop and the proximal hoop is an offset hoop.
